(19) United States
(12) Patent Application Publication
(10) Pub. No.: US 2024/0125805 A1
(43) Pub. Date: Apr. 18, 2024

(54) METHODS FOR POLYPEPTIDE PROCESSING AND ANALYSIS

(71) Applicant: BOARD OF REGENTS, THE UNIVERSITY OF TEXAS SYSTEM, Austin, TX (US)

(72) Inventors: Edward MARCOTTE, Austin, TX (US); Eric ANSLYN, Austin, TX (US); Jagannath SWAMINATHAN, Austin, TX (US); Angela M. BARDO, Austin, TX (US)

(21) Appl. No.: 18/547,687

(22) PCT Filed: Feb. 24, 2022
(86) PCT No.: PCT/US2022/017642

§ 371 Date: Aug. 23, 2023

Related U.S. Application Data
(60) Provisional application No. 63/153,285, filed on Feb. 24, 2021.

(51) Int. Cl. G01N 33/68; G01N 33/58

(52) U.S. Cl. CPC G01N 33/6896; G01N 33/582; G01N 2333/4728; G01N 2800/28

(57) ABSTRACT

Provided herein are methods for analyzing polypeptides and polypeptide complexes. Methods of the present disclosure may be used to identify protein subunits present in a polypeptide, polypeptide complex, or aggregate. These methods may also be used to quantify the subunits (e.g. number of repeating units, protein monomers, repeating domains) in a polypeptide, polypeptide complex, or aggregate.

### CROSS-REFERENCE

This application claims the benefit of U.S. Provisional Application No. 63/153,285, filed Feb. 24, 2021, which is incorporated herein by reference.

### BACKGROUND

Protein aggregation is a common characteristic of many diseases (e.g., neurodegenerative diseases). An abundance of misfolded proteins leading to aggregates and/or oligomers appears to be toxic to cells, leading to cell damage and eventually cell death. In diseases caused by protein aggregation, the severity of the disease often correlates with the expression levels of the aggregates.

For example, accumulation of amyloid-forming proteins may lead to a wide range of diseases known as amyloidoses. Similarly, Alzheimer's disease (AD) neuropathology is characterized by accumulation of amyloid beta protein and/or neurofibrillary tangles comprising tau in the Central Nervous System, synaptic loss, and neuronal death. Specifically, accumulation of amyloid beta as amyloid beta protein plaques or soluble amyloid beta oligomers has been implicated in AD progression.

### SUMMARY

In an aspect, the present disclosure provides a method for analyzing a polypeptide complex from a subject, comprising: (a) providing the polypeptide complex coupled to a capture unit immobilized to a support, wherein the polypeptide complex comprises a plurality of polypeptide molecules; (b) coupling one or more reporter moieties to the polypeptide complex, wherein the one or more reporter moieties comprises a plurality of detectable labels; (c) detecting one or more signals from the plurality of detectable labels; and (d) subjecting the plurality of detectable labels to conditions sufficient to render at most a subset of the one or more detectable labels undetectable.

In some embodiments, the method further comprises (e) detecting a disease or disorder in the subject based at least in part on the one or more signals detected in (c). In some embodiments, the method further comprises repeating (c) and (d) at least once until no signal is detected from the polypeptide complex. In some embodiments, at least a subset of the plurality of polypeptide molecules in the polypeptide complex is quantified.

In some embodiments, a reporter moiety of the one or more reporter moieties is coupled to a polypeptide molecule of the plurality of polypeptide molecules. In some embodiments, a polypeptide molecule of the plurality of polypeptide molecules comprises one or more binding units, wherein at least one binding unit of the one or more binding units is coupled to a reporter moiety of the one or more reporter moieties. In some embodiments, a reporter moiety of the one or more reporter moieties comprises one or more recognition units coupled to at least a subset of the plurality of polypeptide molecules. In some embodiments, the reporter moiety comprises a spacer coupled to a detectable label of the one or more detectable labels. In some embodiments, the one or more signals correspond to the plurality of detectable labels. In some embodiments, the spacer adjoins the detectable label and the recognition unit. In some embodiments, (d) comprises photobleaching a detectable label of the one or more detectable labels. In some embodiments, (d) comprises removing a detectable label of the one or more detectable labels from the polypeptide complex.

In some embodiments, the polypeptide complex comprises at least 2 polypeptide molecules. In some embodiments, the polypeptide complex comprises at least 5 polypeptide molecules. In some embodiments, the polypeptide complex comprises at least 10 polypeptide molecules. In some embodiments, the polypeptide complex comprises at least 20 polypeptide molecules. In some embodiments, the capture unit comprises no more than one antibody.

In some embodiments, the polypeptide complex is a biomarker. In some embodiments, an expression level of the biomarker is indicative of a disease or disorder. In some embodiments, the disease or disorder is Parkinson's disease (PD), Parkinson's disease with dementia (PDD), dementia with Lewy bodies (DLB), multiple system atrophy (MSA), Alzheimer's disease (AD), Pick's disease, frontotemporal dementia (FTD), traumatic brain injury, chronic traumatic encephalopathy (CTE), Huntington's disease, fragile X syndrome, amyotrophic lateral sclerosis (ALS), cryoglobulinemia, amyloidosis, prion disease, transmissible spongiform encephalopathy, or Creutzfeldt-Jakob Disease. In some embodiments, the biomarker is an amyloid protein, an amyloid fibril, an amyloid beta, an amyloid precursor protein, a tau protein, a microtubule-associated protein tau, an alpha synuclein, an immunoglobulin, an islet amyloid polypeptide, a huntingtin protein, a FMRP, a polyglutamine repeat protein, a dipeptide repeat protein, a TDP-43, matrin-3, or a prion. In some embodiments, the biomarker corresponds to a neurodegenerative disease or disorder. In some embodiments, the expression level of the biomarker is quantified and correlated to a health assessment.

In some embodiments, (a) comprises providing the polypeptide complex from a sample from the subject. In some embodiments, the sample comprises cerebrospinal fluid, brain homogenate, tissue homogenate, tissue extract, cell extract, cell homogenate, cell lysate, whole blood, plasma, serum, bodily waste or excretion, or any combination thereof. In some embodiments, the subject's health is assessed based on the detection of the one or more signals detected in (c).

In some embodiments, the support is a bead, a polymer matrix, or an array. In some embodiments, the array is a microscopic slide. In some embodiments, the capture unit is immobilized directly to the support.

In some embodiments, (c) or (d) further comprises providing an energy source. In some embodiments, (c) comprises providing a first energy source sufficient to render the one or more detectable labels optically detectable. In some embodiments, the one or more detectable labels emit an optical signal. In some embodiments, the optical signal is a fluorescent signal. In some embodiments, the first energy source is a light or a laser. In some embodiments, (d) comprises providing a second energy source sufficient to render the at most a subset of the one or more detectable labels undetectable. In some embodiments, the second energy source is a light or a laser. In some embodiments, the first energy source and the second energy source are the same energy source.

In some embodiments, the plurality of polypeptide molecules is homogenous. In some embodiments, the plurality of polypeptide molecules is heterogeneous. In some embodiments, the capture unit is coupled to either the polypeptide complex or an individual polypeptide molecule of the polypeptide complex.

In some embodiments, the polypeptide complex is coupled to the capture unit via a cross-linker. In some embodiments, the cross-linker is an amine specific cross-linker. In some embodiments, the cross-linker is a PEG linker. In some embodiments, the PEG linker is a 1-10 kDa PEG linker. In some embodiments, the PEG linker is a bifunctional biotin PEG linker.

In some embodiments, the method further comprises determining a frequency of polypeptide molecule counts based at least in part on the one or more signals detected in (c). In some embodiments, the method further comprises detecting the disease or disorder in the subject based at least in part on a shift in a distribution of the frequency of polypeptide molecule counts. In some embodiments, the conditions sufficient to render at most a subset of the one or more detectable labels undetectable comprises dye quenching. In some embodiments, the conditions sufficient to render at most a subset of the one or more detectable labels undetectable comprises enzymatic cleavage of the one or more detectable labels.

In another aspect, the present disclosure provides a method for analyzing a polypeptide complex from a subject, comprising: (a) providing the polypeptide complex and one or more reporter moieties coupled thereto, wherein the one or more reporter moieties comprises a plurality of detectable labels, wherein the polypeptide complex comprises a plurality of polypeptide molecules; (b) detecting one or more signals from the plurality of detectable labels; and (c) subjecting the one or more detectable labels to conditions sufficient to render at most a subset of the one or more detectable labels undetectable.

In some embodiments, the method further comprises (d) using at least the one or more signals to quantify an amount of the plurality of polypeptide molecules in the polypeptide complex. In some embodiments, the method further comprises repeating (b) and (c) at least once until no signal is detected from the polypeptide complex. In some embodiments, at least a subset of the plurality of polypeptide molecules in the polypeptide complex is quantified.

In some embodiments, a reporter moiety of the one or more reporter moieties is coupled to a polypeptide molecule of the plurality of polypeptide molecules. In some embodiments, a polypeptide molecule of the plurality of polypeptide molecules comprises one or more binding units, wherein at least one binding unit of the one or more binding units is coupled to a reporter moiety of the one or more reporter moieties. In some embodiments, a reporter moiety of the one or more reporter moieties comprises one or more recognition units coupled to at least a subset of the plurality of polypeptide molecules. In some embodiments, the reporter moiety comprises a spacer coupled to a detectable label of the one or more detectable labels. In some embodiments, the one or more signals correspond to the plurality of detectable labels. In some embodiments, the spacer adjoins the detectable label and the recognition unit.

In some embodiments, (c) comprises photobleaching a detectable label of the one or more detectable labels. In some embodiments, (c) comprises removing a detectable label of the one or more detectable labels from the polypeptide complex. In some embodiments, the polypeptide complex comprises at least 2 polypeptide molecules.

In some embodiments, the polypeptide complex comprises at least 5 polypeptide molecules. In some embodiments, the polypeptide complex comprises at least 10 polypeptide molecules. In some embodiments, the polypeptide complex comprises at least 20 polypeptide molecules. In some embodiments, the capture unit comprises no more than one antibody.

In some embodiments, the method further comprises (e) detecting a disease or disorder in the subject based at least in part on the one or more signals detected in (c). In some embodiments, the polypeptide complex is a biomarker. In some embodiments, an expression level of the biomarker is indicative of a disease or disorder. In some embodiments, the disease or disorder is Parkinson's disease (PD), Parkinson's disease with dementia (PDD), dementia with Lewy bodies (DLB), multiple system atrophy (MSA), Alzheimer's disease (AD), Pick's disease, frontotemporal dementia (FTD), traumatic brain injury, chronic traumatic encephalopathy (CTE), Huntington's disease, fragile X syndrome, amyotrophic lateral sclerosis (ALS), cryoglobulinemia, amyloidosis, prion disease, transmissible spongiform encephalopathy, or Creutzfeldt-Jakob Disease. In some embodiments, the biomarker is an amyloid protein, an amyloid fibril, an amyloid beta, an amyloid precursor protein, a tau protein, a microtubule-associated protein tau, an alpha synuclein, an immunoglobulin, an islet amyloid polypeptide, a huntingtin protein, a FMRP, a polyglutamine repeat protein, a dipeptide repeat protein, a TDP-43, matrin-3, or a prion. In some embodiments, the biomarker corresponds to a neurodegenerative disease or disorder. In some embodiments, the expression level of the biomarker is quantified and correlated to a health assessment.

In some embodiments, (a) comprises providing the polypeptide complex from a sample from a subject. In some embodiments, the sample comprises cerebrospinal fluid, brain homogenate, tissue homogenate, tissue extract, cell extract, cell homogenate, cell lysate, whole blood, plasma, serum, bodily waste or excretion, or any combination thereof. In some embodiments, the subject's health is assessed based on the detection of the one or more signals detected in (b).

In some embodiments, the polypeptide complex is coupled to a capture unit immobilized to a support. In some embodiments, the support is a bead, a polymer matrix, or an array. In some embodiments, the array is a microscopic slide. In some embodiments, the capture unit is immobilized directly to the support.

In some embodiments, (b) and (c) further comprises providing an energy source. In some embodiments, (b) comprises providing a first energy source sufficient to render the one or more detectable labels optically detectable. In some embodiments, the one or more detectable labels emit an optical signal. In some embodiments, the optical signal is a fluorescent signal. In some embodiments, the first energy source is a light or a laser. In some embodiments, (c) comprises providing a second energy source sufficient to render the at most a subset of the one or more detectable labels undetectable. In some embodiments, the second energy source is a light or a laser. In some embodiments, the first energy source and the second energy source are the same energy source.

In some embodiments, the plurality of polypeptide molecules is homogenous. In some embodiments, the plurality of polypeptide molecules is heterogeneous. In some embodiments, the capture unit is coupled to either the polypeptide complex or an individual polypeptide molecule of the polypeptide complex.

In some embodiments, the polypeptide complex is coupled to the capture unit via a cross-linker. In some embodiments, the cross-linker is an amine specific cross-linker. In some embodiments, the cross-linker is a PEG linker. In some embodiments, the PEG linker is a 1-10 kDa PEG linker. In some embodiments, the PEG linker is a bifunctional biotin PEG linker.

In some embodiments, the method further comprises determining a frequency of polypeptide molecule counts based at least in part on the one or more signals detected in (b). In some embodiments, method further comprises detecting a disease or disorder in the subject based at least in part on a shift in a distribution of the frequency of polypeptide molecule counts. In some embodiments, the conditions sufficient to render at most a subset of the one or more detectable labels undetectable comprises dye quenching. In some embodiments, the conditions sufficient to render at most a subset of the one or more detectable labels undetectable comprises enzymatic cleavage of the one or more detectable labels.

Another aspect of the present disclosure provides a method for analyzing a polypeptide complex comprising a plurality of polypeptides of a subject at a single molecule level, comprising detecting an individual polypeptide of the plurality of polypeptides at a sensitivity of at least 60%.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and/or advantages of the present disclosure will be obtained by reference to the following detailed description that sets forth illustrative embodiments, in which the principles of the disclosure are utilized, and the accompanying drawings (also “Figure” and “Fig.” herein), of which:

FIG. 1A schematically illustrates a method for capturing, labeling, and/or detecting polypeptide complexes;

FIG. 1B schematically illustrates a method for counting polypeptide molecules;

FIG. 2 illustrates an example of capturing and/or labeling a polypeptide molecule detection, in accordance with some embodiments;

FIG. 3 illustrates another example of capturing and/or labeling a polypeptide molecule detection, in accordance with some embodiments;

FIG. 4 illustrates another example of capturing and/or labeling a polypeptide molecule, in accordance with some embodiments;

FIG. 5A illustrates an example of capturing and/or labeling a polypeptide complex, in accordance with some embodiments;

FIG. 5B illustrates an example of capturing and/or labeling a polypeptide complex, in accordance with some embodiments;

FIG. 6 illustrates an example of capturing polypeptide molecules and/or a polypeptide complexes, in accordance with some embodiments;

FIGS. 7A and 7B show an example of signal detection, in accordance with some embodiments;

FIGS. 8A-8C show another example of signal detection, in accordance with some embodiments;

FIG. 9 illustrates another example of signal detection, in accordance with some embodiments;

FIG. 10 shows another example of signal detection, in accordance with some embodiments;

FIG. 11 illustrates another example of signal detection, in accordance with some embodiments;

FIG. 12 shows another example of signal detection, in accordance with some embodiments;

FIG. 13 illustrates another example of signal detection, in accordance with some embodiments;

FIG. 14 shows another example of signal detection, in accordance with some embodiments;

FIG. 15 illustrates an example of signal detection, in accordance with some embodiments;

FIG. 16 schematically illustrates an example for antibody screening, in accordance with some embodiments;

FIG. 17 shows a computer system that is programmed or otherwise configured to implement methods provided herein;

FIG. 18A-18B show the effect of slide passivation, which indicates the low non-specific level of multimerized streptavidin/Atto647N-biotin complex.

FIG. 19A-19C show photobleaching and image processing algorithms performed on trimerized streptavidin/alpha-synuclein biotin with detection, which indicated a three-count data.

### DETAILED DESCRIPTION

Provided herein are methods for quantifying components of a biological molecule (e.g., a protein, a biological aggregate, a polypeptide, or a polypeptide complex). Also provided herein are methods for detecting a disease or disorder by quantifying the components of a biological molecule (e.g., a protein, a biological aggregate, a polypeptide, or a polypeptide complex).

Improvements in diagnostic techniques, such as, for example, assays for detecting protein aggregates (oligomers) may advance methods for treating and/or managing diseases or disorders. As recognized herein, improved detection methods may be beneficial for detecting protein aggregates that are toxic to cells and that may cause diseases such as neurodegenerative diseases. Methods to accurately detect and/or quantify protein aggregates may be used to diagnose, identify the stage, and/or find or optimize a treatment for these disease(s).

For example, changes in protein folding may lead to protein accumulation or aggregates in form of amorphous, oligomers, amyloid fibrils, etc. The extent (e.g. quantity, type, and/or quality) of protein aggregation may be correlated with progression or state of a protein conformational diseases or disorders, such as, for example, in prion diseases (e.g., Taupathies, synucleinopathies, etc.) Therefore, techniques that may identify the presence or absence of such protein formations and/or precisely measure the extent of protein misfolding (e.g. number of monomer units in an oligomer) may be instrumental in diagnosing and/or treating such diseases. These techniques may help diagnose, identify the stage of the disease, track progression of the disease, measure effectiveness of various treatments, or optimize treatment regiments.

While various embodiments of the invention have been shown and described herein, it will be obvious to those skilled in the art that such embodiments are provided by way of example only. Numerous variations, changes, and/or substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terminology used herein is for the purpose of describing particular embodiments only and is not intended to be limiting of any embodiment. As used herein, the singular forms “a,” “an” and/or “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

The terms “individual,” “patient,” or “subject” are used interchangeably. None of the terms require or are limited to a situation characterized by the supervision (e.g., constant or intermittent) of a health care worker (e.g., a doctor, a registered nurse, a nurse practitioner, a physician's assistant, an orderly, or a hospice worker). Further, these terms refer to human or animal subjects. These terms may refer to an individual who may be suspected to have a disease or disorder, an individual who may be at risk (e.g. genetic predisposition) to develop a disease or disorder, an individual with a low risk to develop a disorder or disease, or a substantially completely individual. The individual may have a disease or disorder, may be under treatment for a disease or disorder, may be recovering from a disease or disorder, or may be at risk for developing a disease or disorder.

The term “plurality”, as used herein, generally refers to one or more of what the plurality refers to (e.g. molecules or components). Plurality may refer to about at least 2, 3, 4, 5, 6, 7, 8, 10, 11, 12, 13, 14, 15, 16, 17, 18, 19, 20, 21, 22, 23, 24, 25, 30, 40, 50, 100, 1000, 10,000, or more items. For example, a plurality of monomers may refer to one, two, three, four, five, or more monomers.

The terms “polypeptide” or “polypeptide molecule”, as used herein, generally to refer to a polymer of amino acids in which an amino acid may be linked to another amino acid by a peptide bond. In some examples, a polypeptide is a protein. The amino acid may be a naturally occurring amino acid or a non-naturally occurring amino acid (e.g., amino acid analogue). The polymer may be linear or branched and/or may include modified amino acids, and/or may be interrupted by non-amino acids. Polypeptides may occur as single chains or associated chains. The polymer may include a plurality of amino acids and/or may have a secondary and/or tertiary structure (e.g., protein). In some examples, the polymer comprises at least 2, 3, 4, 5, 6, 7, 8, 9, 10, 20, 30, 40, 50, 100, 1000, 10,000, or more amino acids. Polypeptide may be a fragment of a larger polypeptide (e.g. polypeptide complex).

Unless specifically stated or obvious from context, as used herein, the term “about” in reference to a number or range of numbers is understood to mean the stated number and/or numbers +/−10% thereof, or 10% below the lower listed limit and/or 10% above the higher listed limit for the values listed for a range. Alternatively, the term about refers to error inherent in a measurement (e.g., an error associated with an instrument used in measurement, such as a scale or a spectrometer).

The terms “polypeptide complex,” “protein complex,” or “oligomer,” as used herein refers to an arrangement of a plurality of polypeptides (e.g. a protein, a folded polypeptide chain, or misfolded polypeptide chain) in a multi-subunit complex. A polypeptide complex may be considered a quaternary assembly of proteins linked by non-covalent protein-protein interactions. A polypeptide complex may include two or more polypeptide chains (e.g. protein subunits). Polypeptide complexes may comprise one or more polypeptide molecules (e.g. repeating subunits of a single protein or a protein domain). Polypeptide complexes may be homomultimeric (e.g. homooligomers) or heteromultimeric (e.g. heterooligomers) comprising identical subunits, substantially similar, similar, or different subunits, respectively. A polypeptide complex may refer to protein accumulation or aggregates in the form of amorphous, oligomers, or amyloid fibrils. A heterooligomer may be a co-oligomer comprising two or more homooligomers, heterooligomers, or a combination thereof.

The terms “sample”, “biological sample”, “biosample”, or “patient sample”, as used herein, generally refers to a sample containing or suspected of containing a polypeptide (e.g. aggregated proteins, oligomers, etc.) For example, a sample may be a biological sample containing one or more polypeptides. The biological sample may be obtained (e.g., extracted or isolated) from or include blood (e.g., whole blood), cerebrospinal fluid (CSF), plasma, serum, urine, saliva, mucosal excretions, sputum, stool or tears. The biological sample may be a fluid or tissue sample (e.g., cerebrospinal fluid). In some examples, the sample is derived from a homogenized tissue sample (e.g., brain homogenate, liver homogenate, kidney homogenate). In some embodiments, the sample is taken from a specific type of cell (e.g., neuronal cell, muscle cell, liver cell, kidney cell). In some examples, the sample is derived from cerebrospinal fluid. The sample may be acquired from spine via a lumbar puncture, or “spinal tap”. The sample may be acquired from a diseased cell or tissue (e.g., a tumor cell, a necrotic cell), In some examples, the sample is from a disease-associated inclusion (e.g., a plaque, a biofilm, a tumor, a non-cancerous growth). In some examples, the sample is obtained from a patient with a protein conformational disorder (e.g. prion diseases, Taupathies, synucleinopathies).

The term “label” or “detectable label” as used herein generally refers to an agent that generates a measurable signal. Such a signal may include, but is not limited to, fluorescence (e.g., a dye), visible light, mass (e.g., a mass tag), radiation, or a nucleic acid sequence (e.g., a barcode). A “reporter” may comprise a “reporter moiety”. In some cases, the detectable label is a fluorophore. A detectable fluorophore can be, for example, Atto390, Atto425, Atto465, Atto488, Atto495, Atto520, Atto532, AttoRho6G, Atto550, Atto565, AttoRho3B, AttoRho11, AttoRho12, AttoThio12, AttoRho101, Atto590, Atto594, AttoRho13, Atto610, Atto611X, Atto620, AttoRho14, Atto633, Atto647, Atto647N, Atto655, AttoOxa12, Atto665, Atto680, Atto700, Atto725, or Atto740. In some embodiments, the detectable fluorophore is Atto647N. In some cases, a reporter moiety may comprise 1, 2, 3, 4, 5, 6, 7, 8, 9, or 10 detectable labels. In some cases, a reporter moiety may comprise 1 detectable label. In some cases, a reporter moiety may comprise 2 detectable labels. In some cases, a reporter moiety may comprise 2 detectable labels. In some cases, a reporter moiety may comprise 5 detectable labels.

The term “reporter moiety”, as used herein, generally refers to a molecular or macromolecular construct that may couple to another molecule. A reporter moiety may carry a detectable label. The detectable label may provide a detectable signal. The signal may be in the form of fluorescence, phosphorescence, visible light, mass, radiation, or a detectable amino acid sequence. In some cases, the reporter moiety may comprise a protein. In some cases, the reporter moiety may comprise an antibody. In some cases, the reporter moiety may comprise an aptamer. In some cases, the reporter moiety may comprise a molecule carrying a plurality of recognition units and a plurality of detectable labels. In some embodiments, the reporter moiety is an antibody with an affinity for alpha-synuclein, for example, MJFR1. In some embodiments, the reporter moiety is MJFR, wherein the MFR1 is labeled with an Atto647N detectable label.

The term “capture unit”, as used herein, generally refers to a molecule that reacts, binds, or couples to one or more polypeptides (e.g. monomer, oligomer, or target oligomer). A capture unit may comprise one or more capture sites. A capture domain in a polypeptide may bind to the one or more capture sites in a capture unit. A capture unit may be an antibody.

The term “antibody” as used herein generally refers to immunoglobulin molecules and/or immunologically active portions of immunoglobulin molecules. For example, immunoglobulin molecules contain an antigen binding site that specifically binds an antigen. The term may also generally refer to antibodies comprising two immunoglobulin heavy chains and/or two immunoglobulin light chains as well as a variety of forms including full length antibodies and/or functional fragment thereof.

The term “support”, as used herein, generally refers to a solid entity to which a molecular construct may be immobilized. As a non-limiting example, a support may be a bead, a polymer matrix, an array, a microscopic slide, a glass surface, a plastic surface, a transparent surface, a metallic surface, a magnetic surface, a multi-well plate, a nanoparticle, a microparticle, a functionalized surface, or a combination thereof. A bead may be, for example, a marble, a polymer bead (e.g., a polysaccharide bead, a cellulose bead, a synthetic polymer bead, a natural polymer bead), a silica bead, a functionalized bead, an activated bead, a barcoded bead, a labeled bead, a PCA bead, a magnetic bead, or a combination thereof. A bead may be functionalized with a functional motif. Some non-limiting examples of functional motifs include a capture reagent (e.g., pyridinecarboxyaldehyde (PCA)), a biotin, a streptavidin, a strep-tag II, a linker, or a functional group that may react with a molecule (e.g., an aldehyde, a phosphate, a silicate, an ester, an acid, an amide, an alkyne, an azide, an aldehyde dithiolane.

The term “fluorescence”, as used herein, generally refers to the emission of visible light by a substance that has absorbed light of a different wavelength. Fluorescence may provide a non-destructive methods of tracking and/or analyzing biological molecules based on the fluorescent emission at a specific wavelength. Proteins, polypeptide molecules, polypeptide complexes, peptides, nucleic acid, oligonucleotides (including single stranded and/or double stranded primers), or antibodies may be “labeled” with a variety of extrinsic fluorescent molecules referred to as fluorophores.

The term “photobleaching”, as used herein, generally refers to the process of quenching the signal (e.g., fluorescence) of a molecule that emits a radioactive radiation. The photobleaching process may fully quench the signal emitted by the molecule. Photobleaching may occur due to photon-induced chemical damage or covalent modification. Photobleaching can occur when energy transfer from a source of energy (e.g. light, U.V. light, laser) excites a fluorophore molecule to transition from an excited single state to an excited triplet state. The fluorophore may interact with other molecules in the excited triplet state and/or produce irreversible covalent modifications.

The term “quenching”, as described herein, generally refers to a process which decreases the signal intensity of a given substance (e.g., a fluorophore). Quenching may comprise excited state reactions, energy transfer, complex-formation or collisional quenching.

The term “cleavable linker”, as used herein, generally refers to a molecule that can be split into at least two molecules. Non-limiting examples of cleavage methods to split a cleavable unit may include: enzymes, nucleophilic or basic reagents, reducing agents, photo-irradiation, electrophilic or acidic reagents, organometallic or metal reagents, and/or oxidizing reagents.

The terms “cross-linker” or “crosslinking agent,” as described herein generally refers to a molecular construct that couples at least two molecules. A cross-linker may be a molecule which has at least two reactive ends to connect to at least two molecules directly or indirectly. Crosslinking may comprise covalently attaching a protein to another macromolecules (e.g. another protein) or a support. A crosslinker may be reactive toward functional groups on proteins such as, for example. carboxyls, amines, and/or sulfhydryls vione or more reactive groups. Reactive groups of a linker may comprise isothiocyanates, isocyanates, azides, NHS esters (N-Hydroxysuccinimide Esters), sulfonyl chlorides, aldehydes, glyoxals, epoxides, oxiranes, carbonates, aryl halides, imidoesters, carbodiimides, maleimides, haloacetyls, pyridyl disulfides, diazirines, or anhydrides.

The terms “FRET”, or “FET”, as described herein refers to Forster resonance energy transfer. FRET is also known as fluorescence resonance energy transfer (FRET), resonance energy transfer (RET) or electronic energy transfer (EET). FRET process involves energy transfer between two or more molecules, a donor and an acceptor (e.g. dye, chromophore, fluorescent molecule). In FRET energy is transferred non-radiatively (without absorption or emission of photons) from a donor in excited state to an acceptor. FRET can occur when the two molecules are at or closer than a certain distance from one another. Therefore, FRET efficiency can be measured to study molecular distances or localization (e.g. in protein-protein interactions, in protein conformational changes). FRET can also be considered a dynamic quenching mechanism where energy of a donor is quenched by an acceptor molecule.

### Protein Complex Counting

Proteins are the molecular machines of living organisms. When proteins are expressed in the right amounts and/or are folded properly, they may carry on the functions they have in the body. Misfolded proteins and/or protein that are expressed in a biologically inappropriate amounts may not carry their biological functions and/or lead to diseases. A family of diseases associated directly with misfolding of proteins is proteopathy, also known as proteinopathies, protein conformational disorders, or protein misfolding diseases. In proteopathy, often proteins fail to fold into their normal configuration; in this misfolded state, the proteins can become toxic in some way (a gain of toxic function) or they can lose their normal function.

Protein misfolding may lead to abnormally sticky surfaces on a protein that can interact with other proteins or similar misfolded proteins forming aggregates and/or protein complexes. For example, misfolded proteins may have hydrophobic surfaces on their exposed surfaces while hydrophobic moieties may normally be in the core of the proteins. These abnormal protein complexes, interactions, and/or aggregates may render the misfolded protein toxic to the cell, tissue, and/or eventually organs and/or the entire body. For example, in neuronal cells, protein clearance is critical for the maintenance of the integrity of the neurons; abnormal aggregates of misfolded proteins in these cells (e.g. alpha-synuclein, or amyloid beta) may be resistant to protein degradation and/or recycling (e.g. via ubiquitin/proteasome system or autophagy-lysosomal pathway).

In proteopathies, early detection of protein aggregates, abnormal protein interactions or complexes in a patient may help diagnose early onset of the disease. Additionally, quantifying the number of different variations of abnormal complexes and/or aggregates (e.g. protein subunits and/or their counts in homooligomers or heterooligomers) can be instrumental in predicting a stage of the disease, and/or identifying appropriate treatments (e.g. choice of drug(s), intensity, or frequency of the treatment).

The present disclosure provides methods for analyzing polypeptides such as a polypeptide complex (e.g., an oligomer) or polypeptide molecules such as subunits in the polypeptide complex. Methods of the present disclosure may be used to identify the polypeptide complex or the subunits present in a protein complex or an aggregate. The present methods may also be used to quantify an amount of the polypeptide complex or the subunits in an oligomer (e.g. count the number of repeating units, protein monomers, repeating domains). As described elsewhere herein, detecting the presence or absence as well as quantifying the number of subunits in an oligomer may be instrumental in detecting one or more diseases or disorders (e.g. proteopathies) as well as monitoring their progression and/or treatment.

The methods described herein may comprise analyzing a biological sample. The biological sample may comprise a molecule whose presence or absence may be measured or identified. Not meant to be limiting, the biological sample may comprise a macromolecule, such as, for example, a polypeptide or a protein. The biological sample may comprise one or more components (e.g., different polypeptides, heterogenous sample from a CSF of a proteopathy patient). The biological sample may comprise a component of a cell or tissue, a cell or tissue extract, or a fractionated lysate thereof. The biological sample may be substantially purified to contain molecules of a single entity (e.g., a polypeptide, an oligomer, different oligomers of a polypeptide molecule).

Methods consistent with the present disclosure may comprise isolating, enriching, or purifying a biomolecule, biomacromolecular structure (e.g., an organelle or a ribosome), a cell, or tissue from a biological sample. A method may utilize a biological sample as a source for a biological species of interest. For example, an assay may derive a protein, such as alpha synuclein, a cell, such as a circulating tumor cell (CTC), or a nucleic acid, such as cell-free DNA, from a blood or plasma sample. A method may derive multiple, distinct biological species from a biological sample, such as two separate types of cells. In such cases, the distinct biological species may be separated for analysis (e.g., differently sized alpha synuclein clusters may be segregated for separate analyses) or pooled for common analysis. A biological species may be homogenized, fragmented, or lysed prior to analysis. In particular instances, a species or plurality of species from among the homogenate, fragmentation products, or lysate may be collected for analysis. For example, a method may comprise collecting circulating tumor cells from a buffy coat, optionally isolating individual circulating tumor cells, lysing the circulating tumor cells, isolating alpha synuclein clusters from the resulting homogenate, and/or determining the size of the alpha synuclein clusters.

Methods consistent with the present disclosure may comprise nucleic acid analysis, such as sequencing, southern blot, or epigenetic analysis. Nucleic acid analysis may be performed in parallel with a second analytical method, such as an immunohistological interrogation of a peptide complex. The nucleic acid and/or the subject of the second analytical method may be derived from the same subject or the same sample. For example, a method may comprise collecting cell free DNA and/or a peptide complex from a blood sample (e.g., a plasma sample or a buffy coat), subjecting the cell free DNA to nucleic acid analysis (e.g., to identify a cancer marker), and/or subjecting the peptide complex to an immunohistological assay.

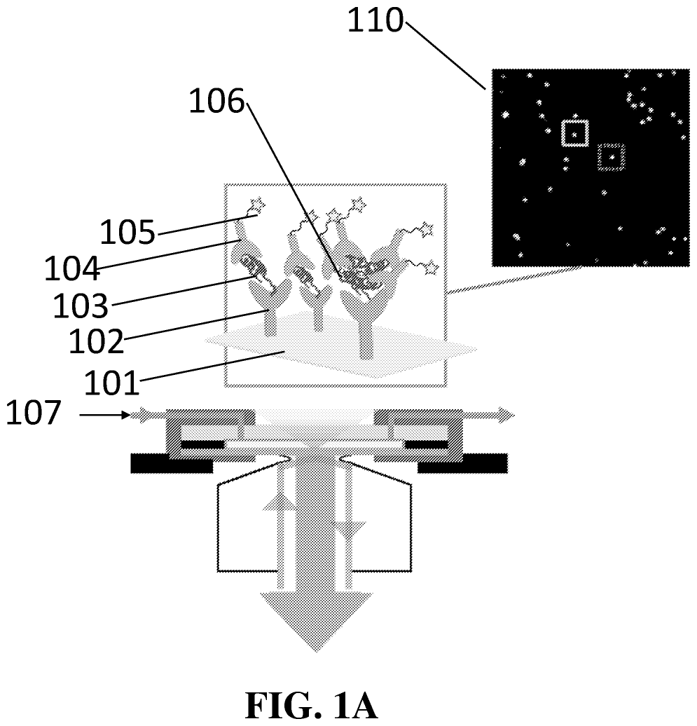

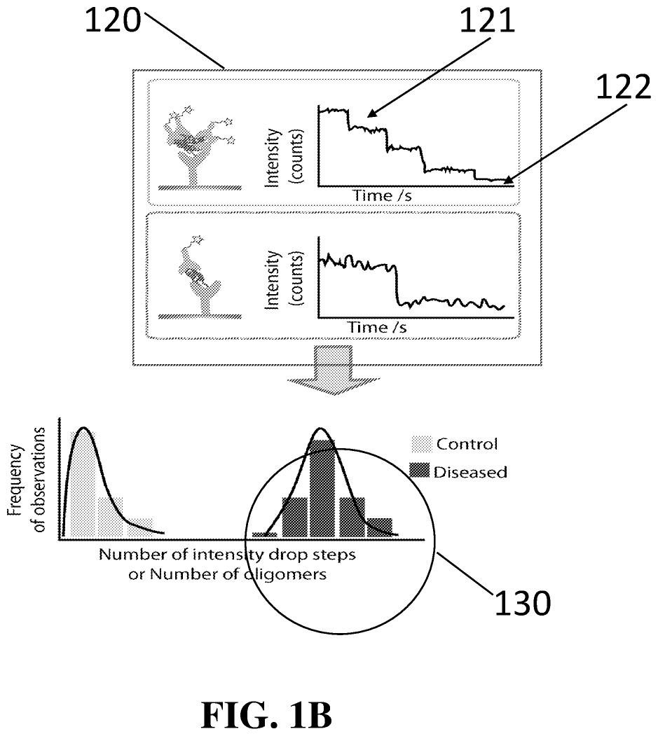

In some cases, polypeptide complexes and/or polypeptide molecules in a sample may be visually detected using a system with a method comprising capturing one or more polypeptide complex or molecule, labeling the one or more polypeptide complex or molecule, and/or detecting the labeled polypeptides. For example, as schematically represented in FIG. 1A, a biological sample (e.g., CSF, blood, saliva) comprising a mixture of proteins may be immobilized on a support 101. The support 101 may, for example, be a glass slide, or a glass slide whose surface has been chemically modified. The support may be modified, for example, by immobilizing a capturing molecule 102 onto a surface of the support 101 to capture one or more molecules of interest such as a polypeptide molecule 103 or a polypeptide complex 106.

The capturing molecule 102 may comprise one or more polyclonal antibodies, monoclonal antibodies, or a combination thereof. One or more reporter moieties 104, carrying one or more detectable labels 105 (e.g., a fluorescent label or a radioactive label), may be configured to bind specifically the one or more molecules of interest (e.g., a polypeptide molecule 103 or a polypeptide complex 106). In some cases, one or more of reporter moieties may be bound to the polypeptide complex 106.

A signal (e.g. fluorescence) of the one or more detectable labels 105 of the reporter moieties 104 bound to the one or more molecules of interest (e.g., single subunit 103 or protein complexes 106) may be detected using an optical device. In some cases, the signal of reporter moieties bound to the one or more molecules of interest distributed across the support 101 can be recorded substantially simultaneously as illustrated in photo 110. The dot boxed by the light solid line illustrates a polypeptide molecule (103) captured by a capturing molecule (102), which is immobilized on the surface of a support (101). The dot boxed by the darker dashed line illustrates a polypeptide complex (106) captured by a capturing molecule (102), which is immobilized on the surface of a support (101).

Suitable optical devices are available that can be applied in this manner. For example, the methods disclosed herein may use a microscope equipped with total internal reflection fluorescence (TIRF) and an intensified charge-couple device (CCD) detector (see Braslaysky, et al., Proc. Nat'l Acad. Sci., 100: 3960-3964 (2003); the reference disclosed herein is incorporated in its entirety). Imaging with a high sensitivity CCD camera allows the instrument to simultaneously record fluorescent intensity of multiple individual protein(s) (e.g. monomers, or oligomers) distributed across a surface. Image collection may be performed using an image splitter that directs light through two band pass filters (one suitable for each fluorescent molecule) to be recorded as two side-by-side images on the CCD surface. Using a motorized microscope stage with automated focus control to image multiple stage positions in the flow cell may allow millions of individual proteins to be detected (e.g. monomers in each oligomer or polypeptide complexes) in an experiment.

Methods provided herein may also comprise quantifying a number of polypeptide molecules in a polypeptide complex such as counting a number of subunits in an oligomer, measuring an extent of polypeptide aggregation, or counting a number of repeating units in protein tandem repeats. FIG. 1B schematically represents a method for quantifying the detected labels. An intensity of the signal from the one or more detectable labels 105 may be used to quantify the number of polypeptide molecules, polypeptide complexes, or a combination thereof. The quantifying of the number of polypeptide molecules or complexes may further comprise eliminating a signal from the detectable labels 120.

A subset of the detectable labels may be rendered undetectable using, for example, photobleaching or by cleaving the detectable labels off from the reporter moieties. The intensity of the signals of a remaining subset of the detectable labels may then be measured and displayed as graph 121. Then, a second subset of the detectable labels may be rendered undetectable followed by measuring the signal intensity of detectable labels. A signal intensity for each detected signal in 110 may be recorded before and/or after rendering a subset of the detectable signals undetectable. This process may be repeated until the measured signal intensity is no more than a baseline or background signal intensity. The baseline or background signal intensity 122 may be a signal intensity measured in a sample that may not be associated with the signal intensity of the reporter moieties bound to the one or more molecules of interest. The top detectable label shows a signal obtained from a sample in FIG. 1A, where a polypeptide molecule (103) is captured by a capturing molecule (102), which is immobilized on the surface of a support (101), which is shown in 110 as a dot boxed by a lighter solid line. The bottom detectable label shows a signal obtained from a sample in FIG. 1A, where a polypeptide complex (106) is captured by a capturing molecule (102), which is immobilized on the surface of a support (101), which is shown in 110 as a dot boxed by a gray dotted line.

A number or frequency of signal quenching steps required to render substantially all detectable labels bound to a molecule of interest (e.g., a polypeptide molecule or polypeptide complex) undetectable may be correlated to a number of subunits (e.g., a polypeptide molecule, a protein repeat subunit in a protein tandem repeat) in the molecule of interest (e.g., a polypeptide molecule, a polypeptide complex such as an protein aggregate or oligomer). As shown in the lower panel of the graph in FIG. 1B, the frequencies of signal quenching observations are different in a control sample (e.g., obtained from a healthy subject) compared to a diseased sample 130. For example, a control sample has very few intensity drop steps or numbers of oligomers compared to the number of intensity drop steps of oligomers of a diseased sample (e.g., sample obtained from a cancer patient). The different frequencies of signal quenching observations may be used to diagnose a disease or condition or a severity or state thereof.

### Polypeptide Analysis

Provided herein are methods for analyzing and/or quantifying polypeptides. A method disclosed herein may be used to analyze a polypeptide complex comprising a plurality of polypeptides of a subject at a single molecule level, comprising detecting an individual polypeptide of the plurality of polypeptides at a sensitivity of at least 60%. In some cases, a method of the disclosure may be used to quantify a plurality of polypeptides of a subject at a single molecule level, comprising detecting an individual polypeptide of the plurality of polypeptides at a sensitivity of at least 60%. In some cases, the methods disclosed herein can have a sensitivity of at least about 50%, at least about 60%, at least about 70%, at least about 80%, at least about 85%, at least about 90%, or at least about 95%.

A polypeptide complex comprising one or more of polypeptide molecules may be immobilized to a support via a capture unit. In some cases, the polypeptide complex may be coupled to the capture unit via a cross-linker. Furthermore, one or more of reporter moieties comprising one or more of detectable labels may be coupled to the one or more of polypeptide molecules. Signals corresponding the one or more of detectable labels attached to the one or more of polypeptide complex may be detected with suitable methods. Moreover, photo bleaching the one or more of detected labels under sufficient conditions is performed so that at most a subset of the one or more of detectable labels undetected. In additional, photo bleaching may be repeated until signals corresponding to the one or more of detected labels coupled to the polypeptide complex cannot be detected.

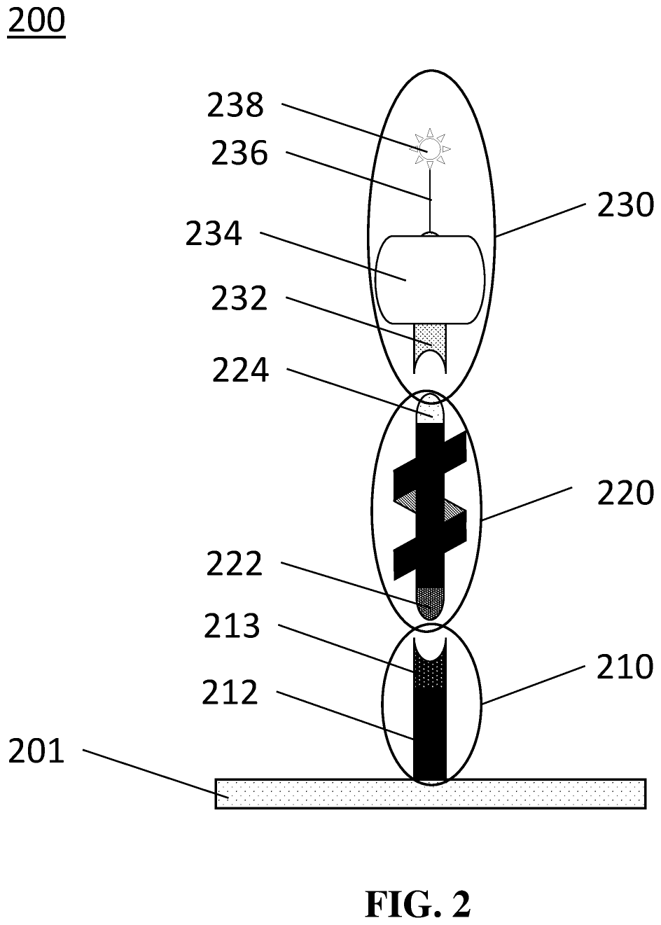

FIG. 2 illustrates an example of capturing and/or labeling a polypeptide molecule according to some embodiments of the methods disclosed herein. The polypeptide molecule 220 may be coupled to a capture unit 210 via a capture site 213 on the capture unit 210 and/or a capture domain 222 on the polypeptide molecule 220. The reporter moiety 230 may be coupled to the polypeptide molecule 220 via a binding unit 224 on the polypeptide molecule 220 and/or a recognition unit 232 on the reporter moiety 230. The reporter moiety 230 may further comprise a reporter molecule 234 and/or a detectable label 238. The reporter moiety 230 may also comprise a spacer 236, wherein the spacer is coupled to the reporter molecule 234 and/or the detectable label 238.

A spacer may be used to adjoin the reporter moiety and a polypeptide molecule (e.g., a monomer, or a polypeptide molecule in an oligomer). A spacer may position two entities such as two detectable labels at a distance from one another in order to optimize a functionality (e.g. FRET). The distance may prevent signal masking or quenching one another. The distance may promote FRET. A spacer may be implemented to prevent crowding or steric interference. A spacer can be composed of a polymer, a biopolymer, or a non-polymer, a heteroatomic chain, a polyamine chain, a polyester chain, a polyether chain, or a polyamide chain.

The capture unit 210 may be a molecule that can be coupled to a polypeptide. The capture unit 210 may be an antibody. The capture unit 210 can be coupled to a support 201. The capture unit 210 can be bound to the support 201 by non-covalent interactions (e.g., Hydrophobic, van der Waals, and/or pi-pi interactions) or covalent interactions (212). The capture unit 210 may comprise, for example, an immunoglobulin molecule, a polyclonal antibody, a monoclonal antibody, a recombinant antibody, a chimeric antibody, a humanized antibody, a CDR-grafted antibody, F(ab)2, Fv, scFv, IgGACH2, F(ab′)2, scFv2CH3, F(ab), VL, VH, scFv4, scFv3, scFv2, dsFv, Fv, scFv-Fc, (scFv)2, a disulfide linked Fv, a single domain antibody (dAb), a diabody, a multispecific antibody, a dual specific antibody, an anti-idiotypic antibody, a bispecific antibody, any isotype (including, without limitation IgA, IgD, IgE, IgG, or IgM) a modified antibody, and/or a synthetic antibody (including, without limitation non-depleting IgG antibodies, T-bodies, or other Fc or Fab variants of antibodies). The capture unit may comprise one or more antibodies. The capture unit may comprise no more than one antibody. Methods described herein may comprise one or more of different capture units, such as different antibodies.

The capture unit 210 can be coupled to a support 201. The support 201 may be a bead, a polymer matrix, an array, or any combination thereof. The support can be a slide. The slide can be a microscopic slide suitable for single molecule imaging. The support (e.g., slide) can comprise a surface. The surface can be functionalized using a functional group to promote coupling of the capture unit to the support. The functional group may comprise amines, sulfhydryls, acids, alcohols, bromides, maleamides, succinimidyl esters (NHS), sulfosuccinimidyl esters, disulfides, azides, alkynes, isothiocyanates (ITC), or combinations thereof. The support can be functionalized using an azide, an amine, a biotin, or a combination thereof. The support can be functionalized using an azide. The support can be functionalized using an amine. The support can be functionalized using a biotin. The support may comprise protected functional groups, such as, for example, Boc, Fmoc, alkyl ester, Cbz, or combinations thereof.

The support may be a solid support or a semi-solid support. The solid support or semi-solid support may be a bead. The bead may be a gel bead. The bead may be a polymer bead. The support may be a resin. Non-limiting supports may comprise, for example, agarose, sepharose, polystyrene, polyethylene glycol (PEG), or any combination thereof. The support may be a polystyrene bead. The support may be a PEGA resin. The support may be an amino PEGA resin. The bead may contain a metal core. The bead may be a polymer magnetic bead. The polymer magnetic bead may comprise a metal-oxide. The support may comprise at least one iron oxide core.

The capturing unit (e.g., an antibody) can be immobilized to the support via non-covalent interactions (e.g., Hydrophobic, van der Waals, and/or pi-pi interactions) or covalent interactions Immobilization using non-covalent interactions may include passive adsorption also known as passivation. The support may comprise a surface passivated using PEG or bovine serum albumin (BSA) Immobilization using non-covalent interactions may include a biotin-streptavidin system.

The capture unit (e.g., an antibody) may be immobilized to the support via covalent interactions. Immobilization using covalent interactions may include cross-linking. A cross-linker or cross-linking agent may comprise at least two reactive groups; at least one reactive group may bind to the support, while at least one other reactive group can bind the polypeptide molecule substantially simultaneous. The reactive groups in a cross-linker may comprise isothiocyanates, isocyanates, azides, NHS esters (N-Hydroxysuccinimide Esters), sulfonyl chlorides, aldehydes, glyoxals, epoxides, oxiranes, carbonates, aryl halides, imidoesters, carbodiimides, maleimides, haloacetyls, pyridyl disulfides, diazirines, or anhydrides. In some embodiments, the cross-linker comprises dissucinimidyl sulfoxide (DSSO). Another non-limiting example of covalent immobilization may include click chemistry (e.g., an azide can react with an alkyne to form a five-membered heteroatom ring in the presence of copper). Other non-limiting examples of immobilizing the capture unit 210 (e.g., antibody) to the support 201 may include Carbohydrate-binding, Molecular imprinting, Ig binding peptide, Calixarene derivatives, Material binding peptide, or a combination thereof.

The capture units may be coupled to the support in a predetermined density (˜4000 molecules/[200×200 μm²]). In every 1 squared millimeter of surface area of a support there may be about 1000 to 10,000,000 capture units (e.g., antibody molecule) attached thereto. The density of the capture unit molecules on the support surface may be at least about 1000, 5000, 10000, 20000, 40000, 60000, 80000, 100000, 150000, 200000, 250000, 300000, 350000, 400000, 450000, 500000, 550000, 600000, 650000, 700000, 750000, 800000, 850000, 900000, 950000, 1000000, or more molecules per square millimeter. The density of the capture unit molecules on the support surface may be at most about 1000000, 950000, 900000, 850000, 800000, 750000, 700000, 650000, 600000, 550000, 500000, 450000, 400000, 350000, 300000, 250000, 200000, 150000, 100000, 80000, 60000, 40000, 20000, 10000, 5000, 1000 or less molecules per square millimeter. The density of the capture unit molecules on the support surface may be between about 1000 molecules per square millimeter to about 1000000 molecules per square millimeter. In some embodiments, the density of the capture unit molecules on the support surface is from about 1000 molecules to about 2000 molecules per 200 μm×200 μm area.

The methods provided herein may comprise providing a polypeptide complex comprising one or more of polypeptide molecules. The polypeptide complex may comprise misfolded proteins or protein aggregates (e.g., alpha-synuclein aggregates, oligomers, amyloid fibrils). The misfolded proteins or protein aggregates may cause a disease and/or disorder in a subject. Polypeptide complexes may comprise one or more polypeptide molecules (e.g. repeating subunits a single protein or a protein domain). Polypeptide complexes may be homomultimeric (e.g. homooligomers) or heteromultimeric (e.g. heterooligomers). The polypeptide complex may be an oligomer. The oligomer may be a homooligomer comprising similar polypeptide molecules. The oligomer may be a heterooligomer comprising different polypeptide molecules. The oligomer may comprise one or more of polypeptide molecules (e.g., monomer, repeating unit, protein subunit). The oligomer may include at least 2, 3, 4, 5, 6, 7, 8, 10, 11, 12, 13, 14, 15, 16, 17, 18, 19, 20, 21, 22, 23, 24, 25, 30, 40, 50, 100, or more polypeptide molecules. The oligomer may include at most 100, 50, 40, 30, 25, 24, 23, 22, 21, 20, 19, 18, 17, 16, 15, 14, 13, 12, 11, 10, 8, 7, 6, 5, 4, 3, or 2 polypeptide molecules. The polypeptide complex may comprise at least 2 polypeptide molecules. The polypeptide complex may comprise at least 5 polypeptide molecules. The polypeptide complex may comprise at least 10 polypeptide molecules. The polypeptide complex may comprise at least 20 polypeptide molecules.

In some cases, the polypeptide complex can comprise at least 20 polypeptide molecules. In some cases, the polypeptide complex may be a biomarker. In some cases, the biomarker may be indicative of a disease or a disorder. In some cases, the disease or disorder is a neurogenerative disease or a synucleinopathy. In some cases, the disease or disorder may include Parkinson's disease (PD), Parkinson's disease with dementia (PDD), dementia with Lewy bodies (DLB), multiple system atrophy (MSA), Alzheimer's disease (AD), Pick's disease, frontotemporal dementia (FTD), traumatic brain injury, chronic traumatic encephalopathy (CTE), Huntington's disease, fragile X syndrome, amyotrophic lateral sclerosis (ALS), cryoglobulinemia, amyloidosis, prion disease, transmissible spongiform encephalopathy, or Creutzfeldt-Jakob Disease. In some cases, the disease or disorder may include a synucleinopathy associated with aggregation of alpha-synuclein or formation of alpha-synuclein oligomers within cells. In some cases, the methods disclosed herein may modulate interactions between alpha-synuclein and lipids. Please see Killinger, Bryan A., et al. “Endogenous alpha-synuclein monomers, oligomers and resulting pathology: Let's talk about the lipids in the room.” npj Parkinson's Disease 5.1 (2019): 1-8, which is incorporated herein by reference.

In some cases, the disease or disorder may be a cancer. Aberrant α-, β-, and/or γ-synuclein expression can manifest in a wide range of cancers, including a wide range of carcinomas, gangliogliomas, medulloblastomas, neuroblastomas, neurocytomas, breast, and/or esophageal cancers. Synuclein expression can also contribute to metastasis, and thus can serve as a useful marker for cancer progression. Accordingly, a method of the present disclosure may comprise analyzing a cell or tissue sample to identify a cancer state in a subject. The method may comprise isolating or enriching the cell from a biological sample, such as a blood or tissue sample. A number of methods of the present disclosure not only enable cancer type identification, but also cancer stage identification. For example, the stages of some cases of pineoblastoma may be distinguished by the degree of synuclein overexpression.

An example of a method consistent with the present disclosure comprises isolating or enriching a circulating tumor cell (CTC) from a blood sample, optionally lysing the circulating tumor cell, and/or determining the type or stage of cancer of the circulating tumor cell by analyzing a protein or protein complex from the circulating tumor cell (or lysate thereof). A method may also comprise isolating or enriching a cell (e.g., a circulating tumor cell) from a biological sample, analyzing a protein or protein complex disposed on the surface of the cell, and/or identifying a disease state or stage based on the analysis. A method may comprise collecting two biological species (e.g., two different types of cells) from a single sample. In some cases, the first biological species is associated with a disease state and the second biological species is associated with a healthy (e.g., non-cancerous or non-Alzheimer's) state.

A method may also comprise collecting (e.g., isolating) a first biological species from a first biological sample and collecting a second biological species from a second biological sample. For example, a cancer assay may comprise collecting a circulating tumor cell from a patient's blood sample and a healthy cell from non-cancerous tissue. The first biological species and second biological species may be separately analyzed, and/or the analysis of one or both of the species may be used to identify a disease state (e.g., a type or a stage of a disease).

The methods provided herein may comprise providing a polypeptide complex comprising one or more polypeptide molecules and one or more reporter moieties coupled thereto, wherein the one or more reporter moieties comprises one or more detectable labels, and/or wherein the polypeptide complex is coupled to a capture unit. The methods provided herein may comprise providing a polypeptide complex comprising one or more of polypeptide molecules and one or more of reporter moieties coupled thereto, wherein the one or more of reporter moieties comprises one or more of detectable labels, and/or wherein the polypeptide complex is coupled to a capture unit.

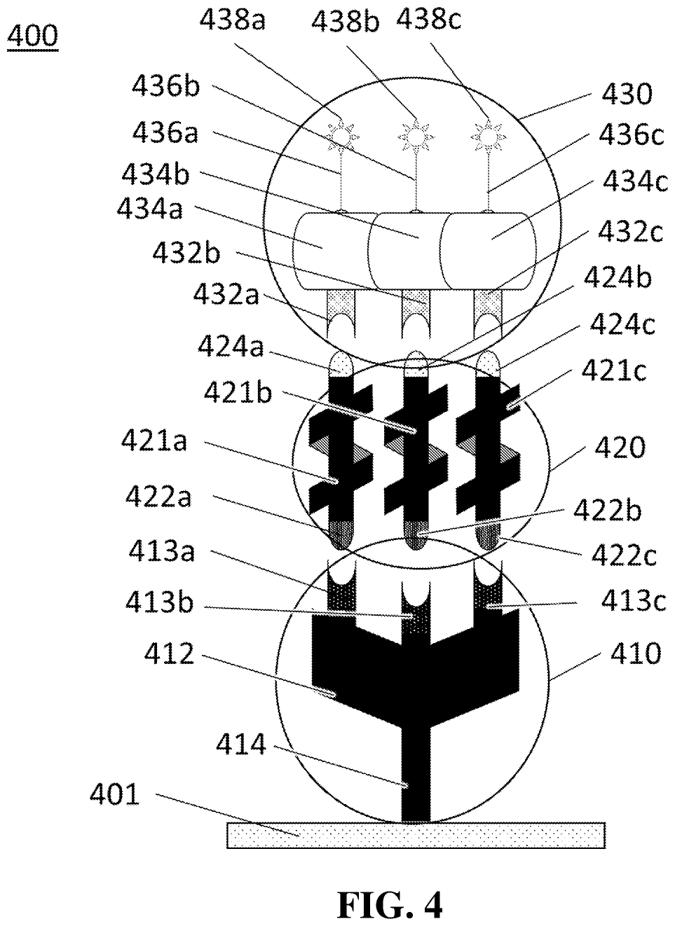

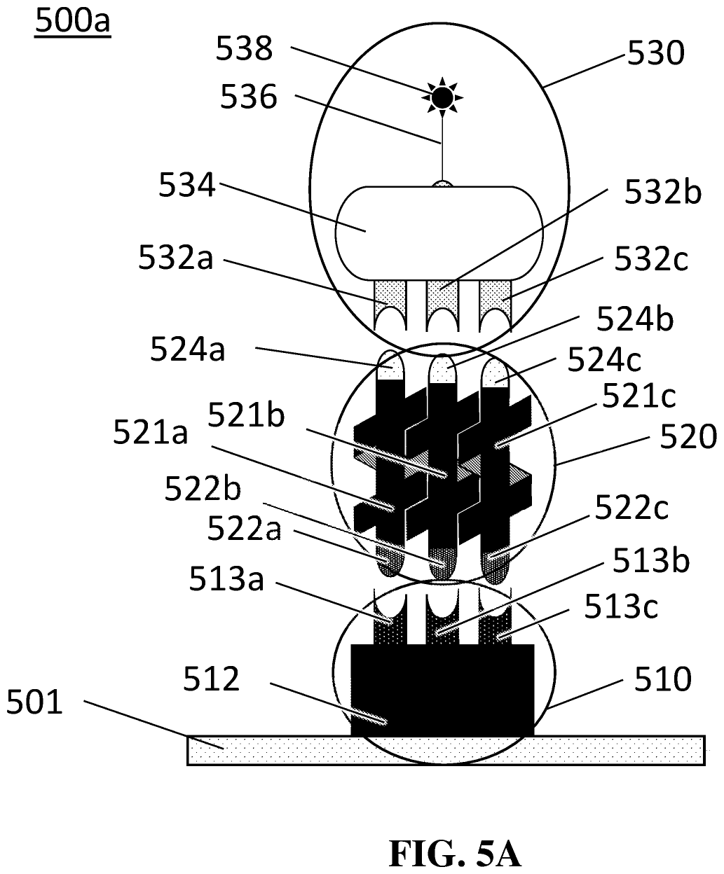

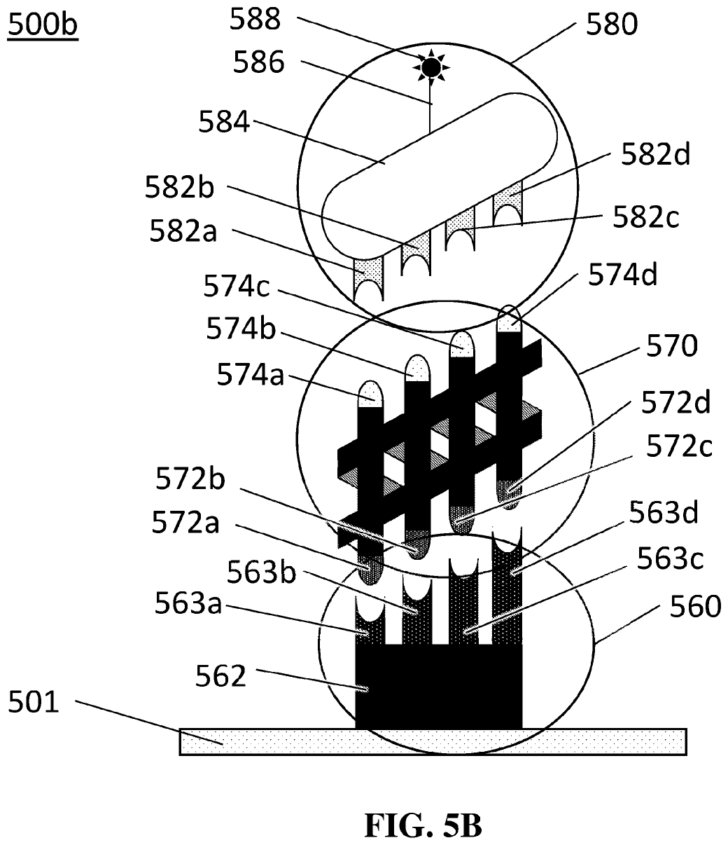

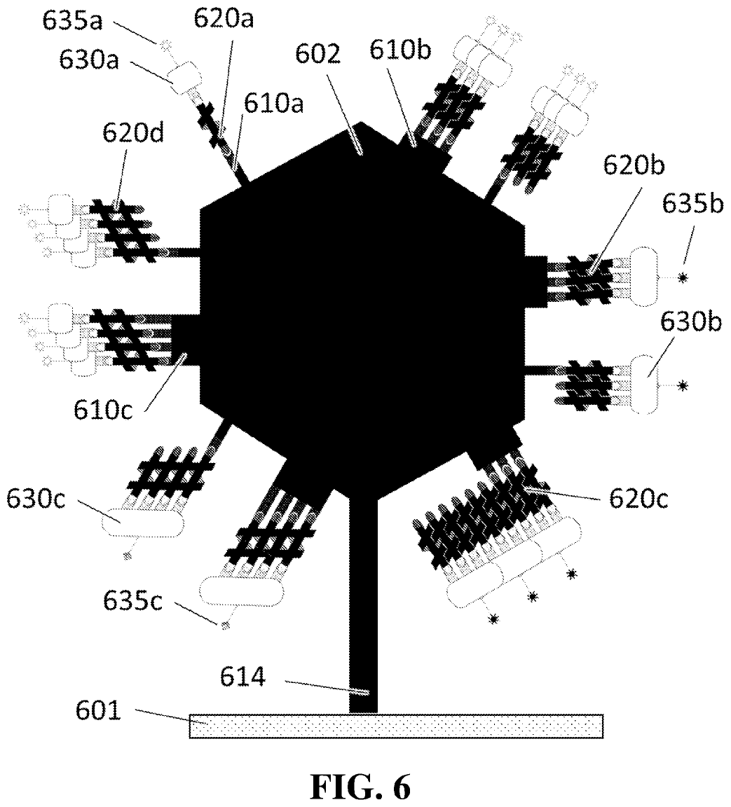

The capture site may be configured to bind specifically to a capture domain, for example, a polypeptide molecule (e.g., monomer) or a polypeptide complex (e.g., oligomer). The capture site may be configured to bind to one or more of areas (e.g., polyclonal antibody) on a polypeptide molecule (e.g., monomer) or polypeptide complex (e.g., oligomer). The capture site may be configured to bind to a predetermined area (e.g., monoclonal antibody) on a polypeptide molecule (e.g., monomer) or polypeptide complex (e.g., oligomer). The capture unit may comprise one or more of capture sites. The capture unit may comprise at least about 1, 2, 3, 4, 5, 6, 7, 8, 10, 11, 12, 13, 14, 15, 16, 17, 18, 19, 20, or more capture sites. The capture unit may comprise at most about 20, 19, 18, 17, 16, 15, 14, 13, 12, 11, 10, 8, 7, 6, 5, 4, 3, 2, or 1 capture sites. The plurality of capture sites in a capture unit may be similar capture sites or they may be different. As shown in FIG. 4, a plurality of similar capture sites 413a-c in a capture unit 410 may couple to similar capture domains 422a-c in one or more polypeptide molecules 421a-c; or as shown in FIG. 5A, a plurality of similar capture sites 513a-c in a capture unit 510 may couple to similar capture domains 522a-c in one or more polypeptide molecules (e.g., monomers) 521a-c in an oligomer 520. A plurality of different capture sites in a capture unit may couple to different capture domains (e.g., different polypeptide molecules, different monomers in an oligomer (FIG. 6, capture site 610 and/or 620)).

The plurality of capture sites in a capture unit may be different capture sites. The plurality of capture sites in a capture unit may bind to a similar capture domain. The plurality of capture sites in a capture unit may bind to different capture domains respectively. In some cases, a capture domain in a polypeptide may bind to one or more capture sites in a capture unit, and the capture unit may be an antibody. In some cases, a capture domain may bind one or more antigen binding sites of an antibody. In some cases, a capture domain may bind two or more antigen binding sites of an antibody, wherein the antigen binding sites are similar and bind the same antigen species. In some cases, a capture domain may bind two or more antigen binding sites of an antibody, wherein the antigen bindings sites of the antibody are different antigen binding sites. In some cases, a first capture unit may have more capture sites than a second capture unit. In some cases, a plurality of capture units may comprise different numbers of capture sites. The polypeptide molecule or complex and the capture unit may be cross-linked using cross-linking agents. The crosslinking may be performed in predetermined conditions including temperature, incubation time, etc. The crosslinking may be performed at room temperature. A predetermined incubation time may be required for cross-linking process to be performed substantially completely. The predetermined incubation time may be about 1 minute (min) to 5 min, 5 min to 10 min, 10 min to 15 min, 15 min to 20 min, 20 min to 30 min, 30 min to 60 min, or a time period beyond or between thereof.

The methods provided herein may comprise providing a polypeptide complex comprising one or more of polypeptide molecules and one or more of reporter moieties coupled thereto, wherein the one or more of reporter moieties comprises one or more of detectable labels. The reporter moiety (e.g., 230, 330, 430, 530, 630, 830, 930, 1130, 1230, 1330, or 1430 of FIGS. 2-6, 8, 9, and 11-14) may comprise a reporter molecule (e.g., 234, 334, 434, 534, 634, or 705 of FIG. 2-7) carrying a recognition unit (e.g., 232, 332, 432, 532, or 632 of FIG. 2-6) and a detectable label (238, 338, 438, 538, 638, 710, 840, 938, 1030, 1135, 1235, 1335, or 1435 of FIGS. 2-6, 8, 9, and 11-14). The detectable label may be directly coupled to the reporter moiety. The reporter moiety may further comprise a spacer (e.g., 236, 336, 436, 536, or 706 of FIGS. 2-5 and 7) wherein the detectable label may be coupled to the reporter moiety via the spacer. The spacer may adjoin the detectable label and the recognition unit. The reporter moiety may comprise a protein. The reporter moiety may comprise an antibody. The reporter moiety may comprise a molecule carrying one or more of recognition units and one or more of detectable labels.

The one or more of recognition units in a reporter moiety may allow binding to one or more of binding units on one or more polypeptide molecules to increase binding strength. In some cases, the one or more of recognition units in a reporter moiety may bind to different binding units on one or more polypeptide molecules. This may allow for recognizing and/or further labeling specific polypeptide complexes that comprise polypeptide molecules with the binding units recognizable to the one or more of recognition units in a reporter moiety. The one or more of different recognition units in a reporter moiety may also bind to and/or further label one or more of polypeptide molecules with different binding units. This may be used to label polypeptide molecules in a heterogenous sample comprising a mixture of different polypeptide molecules and/or polypeptide complexes. In some cases, a reporter moiety may comprise at least 1, at least 2, at least 3, at least 4, at least 5, at least 6, at least 7, at least 8, at least 9, or at least 10 recognition units. In some embodiments, a reporter moiety comprises at least 1 recognition unit. In some embodiments, a reporter moiety comprises at least 2 recognition units. In some cases, a reporter moiety may comprise at least 1, at least 2, at least 3, at least 4, at least 5, at least 6, at least 7, at least 8, at least 9, or at least 10 reporter molecules, each of which comprise at least 1, at least 2, at least 3, at least 4, at least 5, at least 6, at least 7, at least 8, at least 9, or at least 10 recognition units. In some cases, a reporter moiety may comprise 1 reporter molecule, wherein the 1 reporter molecule comprises at least 1, at least 2, at least 3, at least 4, at least 5, at least 6, at least 7, at least 8, at least 9, or at least 10 recognition units. In some cases, a reporter moiety may comprise 1 reporter molecule, wherein the 1 reporter molecule comprises 1 recognition unit. In some cases, a reporter moiety may comprise 1 reporter molecule, wherein the 1 reporter molecule comprises 3 recognition units. In some cases, a reporter moiety may comprise 1 reporter molecule, wherein the 1 reporter molecule comprises 5 recognition units. In some cases, a reporter moiety may comprise 3 reporter molecules, wherein each of the 3 reporter molecules comprises 1 recognition unit. In some cases, a reporter moiety may comprise 5 reporter molecules, wherein each of the 5 reporter molecules comprises 1 recognition unit. In some cases, a reporter moiety may comprise 10 reporter molecules, wherein each of the 10 reporter molecules comprises 1 recognition unit. In some cases, a reporter moiety may comprise 10 reporter molecules, wherein each of the 10 reporter molecules comprises 5 recognition units.

The one or more of detectable labels in a reporter moiety may be used to count the number of polypeptide molecules in each polypeptide complex. For example, the one or more of detectable labels may be rendered undetectable one by one in one or more of steps; and/or an intensity of a signal from the one or more of detectable labels may be measured before rendering the label(s) undetectable at each step. The number of steps required to render the labels undetectable may be used to correlate a number of polypeptide molecules in a sample, where the sample comprises polypeptide complexes. The one or more of detectable labels may also be used for labeling and/or further detecting one or more of similar or different molecules of interest simultaneously, where the molecules of interest may be a polypeptide molecule or a polypeptide complex.

A reporter moiety may emit a signal upon excitation. Excitation may be provided in the form of electromagnetic radiation (e.g., light). A reporter moiety may also decrease or lose signal upon excitation. A signal emitted from a reporter moiety (or detectable label disposed thereon) may be detectable. A signal may be optical, chemical, radiometric, electronic, informational, or a combination thereof. An optical signal may be luminescent (e.g., chemiluminescent, bioluminescent, electroluminescent, sonoluminescent, photoluminescent, radioluminescent, or thermoluminescent. Some examples of photoluminescent optical signals include fluorescent or phosphorescent signals. An optical signal may come from a chromophore (e.g., a fluorophore, fluorescent dye). An optical signal may be any molecule, macromolecule, or molecular construct capable of emitting photons. Optical signals may be emitted in response to excitation. Optical signals may be differentiable from one another, such as by color. In some examples, it is advantageous to use multiple optical signals within a single system, method, or kit. For example, it may be advantageous to provide one or more of fluorophores, some or all of which being capable of emitting a differentiable optical signal. A plurality of optical signals may include, for example, multiple colors. It may be advantageous to provide fluorescent dyes that produce one color, two colors, three colors, four colors, five colors, or more. It may be advantageous to provide fluorescent dyes that produce twenty colors or more. Fluorophores may comprise one or more classes of dyes such as rhodamine or Atto647N. Fluorophores may include, for example, a fluorophore-iodoacetamide (e.g., Atto647N-Iodoacetamide); a fluorophore-succinimidyl ester (e.g., Atto647N-NHS), a fluorophore-amine (e.g., Atto647N-Amine), a dithiolane-fluorophore (e.g. a custom synthesized fluorophore, an oxidized dithiolane-fluorophore, a reduce dithiolane-fluorophore), a fluorophore-Azide (e.g., Atto647N-Azide), Oregon Green (OG)-iodoacetamide, OG488-NHS, OG488-Azide, OG488-Tetrazine, OG514-NHS, Janelia Fluor (JF)-NHS, JF-FreeAcid, JF-Azide, JF-Dithiolane, Atto647N-Alkyne, Atto647N-FreeAcid, Atto425-NHS, Atto425-FreeAcid, Atto425-Amine, Atto425-Azide, Atto425-DBCO, SF554-NHS, or TexasRed-NHS. Optical signals may also comprise an absence or a loss of an optical signal (e.g., photobleaching, photoquenching) or a change in optical signal (e.g., FRET, BRET, homo-FRET, or other energy transfer luminescence, such as Alexa fluors, BODIPY dyes, Xanthene dyes, or Cyanine dyes).

In some cases, a method of the disclosure may comprise providing a polypeptide complex comprising a capture unit, a polypeptide molecule, and a reporter moiety. In some cases, the capture unit may be coupled to a support and/or comprise a capture site, which binds to a capture domain of the polypeptide molecule. In some cases, the polypeptide may comprise a capture domain that binds to a capture site of a capture unit. In some cases, the polypeptide may comprise a binding unit that binds to a recognition unit of a reporter molecule. In some cases, a reporter moiety may comprise a reporter molecule that comprises a recognition unit that binds to a polypeptide molecule. In some cases, a reporter molecule may further comprise a detectable label that is coupled to the reporter molecule, for example, by a covalent bond.

As illustrated in FIG. 2, the polypeptide 220 (e.g., polypeptide molecule, polypeptide complex) can be coupled to the capture unit 210 via the capture site 213 on the capture unit 210 and/or the capture domain 222 on the polypeptide molecule 220. Method 200 can use recognition unit 232, which may be configured to couple to a binding unit 224 in the polypeptide molecule 220. The reporter moiety 230 may comprise a recognition unit 232 configured to couple to at least one or more polypeptide molecules in a polypeptide complex. The reporter moiety 230 may be coupled to the polypeptide molecule 220 via a binding unit 224 on the polypeptide molecule 220 and/or a recognition unit 232 on the reporter moiety 230. The reporter moiety 230 may further comprise a reporter molecule 234 and/or a detectable label 238. The reporter moiety 230 may also comprise a spacer 236, wherein the spacer is coupled to the reporter molecule 234 and/or the detectable label 238.

In some cases, a method of the disclosure may comprise providing a polypeptide complex comprising a capture unit, a polypeptide molecule comprising a plurality of capture domains and/or a plurality of binding units, and a reporter moiety. In some cases, the capture unit may be coupled to a support and/or comprise a capture site, which binds to one of a plurality of capture domains of the polypeptide molecule. In some cases, the polypeptide may comprise a plurality of capture domains, one of which binds to a capture site of a capture unit. In some cases, the polypeptide may comprise a plurality of binding unit that binds to a recognition unit of a reporter molecule. In some cases, a reporter moiety may comprise a reporter molecule comprising a recognition unit that binds to a polypeptide molecule comprising a plurality of binding units. In some cases, a reporter molecule may further comprise a detectable label that is coupled to the reporter molecule, for example, by a covalent bond.

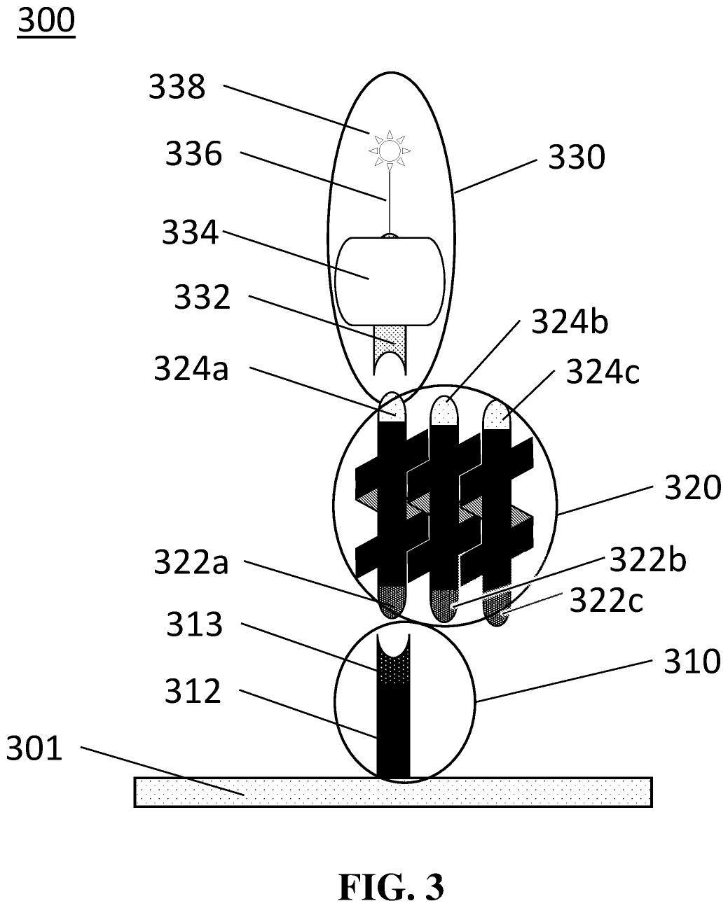

As shown in FIG. 3, method 300 may comprise a support 301, a capture unit 310, a polypeptide complex 320, and/or a reporter moiety 330. The polypeptide complex 320 may comprise at least one capture domain, for example, 322a, 322b, or 322c. The polypeptide complex 320 may comprise a capture molecule 312 and/or a plurality of capture domains 322a-c and/or a plurality of binding units 324a-c. The capture unit 310 may comprise a capture site 313. The capture site 313 may be configured to specifically couple to at least one capture domain 322a, 322b, or 322c in polypeptide complex 320. The capture site 313 may be configured to couple to one capture domain or more than one capture domain, for example, 322a, 322b, or 322c. Similarly, the reporter moiety 330 may comprise a recognition unit 332 that is configured to couple to at least one of the plurality of binding units 324a-c in the polypeptide complex 320. The recognition unit 332 may be configured to couple to one binding unit in the polypeptide complex 320. One or more reporter moieties similar to reporter moiety 330 may bind to one or more binding units from the plurality of binding units that may be available for binding (e.g., 324b-c). The reporter moiety 330 may comprise a detectable label 338 coupled to a reporter molecule 334 via a crosslinker 336. The detectable label and/or the recognition unit may be coupled to the reporter molecule without using a crosslinker. The reporter moiety 330 may comprise an antibody. The reporter moiety 330 may be oligomeric (e.g., comprise multiple coupled reporters, such as multiple fluorophore-labeled antibodies).

In some cases, a method of the disclosure may comprise providing a polypeptide complex comprising a capture unit, a polypeptide molecule, and a reporter moiety. In some cases, the capture unit may be coupled to a support and/or comprise a plurality of capture sites, which bind to a plurality of capture domains of the polypeptide molecule. In some cases, the capture unit may be attached to a solid support via a covalently bonded cross-linker. In some cases, the polypeptide may comprise a plurality of capture domains that bind to a plurality of capture sites of a capture unit. In some cases, the polypeptide may comprise a plurality of binding units that bind to a recognition unit of a reporter molecule. In some cases, a reporter molecule may comprise a recognition unit that binds to a polypeptide molecule. In some cases, a reporter molecule may further comprise a detectable label that is coupled to the reporter molecule, for example, by a covalent bond. In some cases, a reporter moiety may comprise a plurality of reporter molecules, a plurality of recognition units, and a plurality of detectable labels.

FIG. 4 illustrates another embodiment of the methods disclosed herein. Method 400 may comprise a support 401, a capture unit 410, a plurality of polypeptide molecules 420, and/or a reporter moiety 430. The plurality of polypeptide molecules may comprise one or more similar polypeptide molecules. The plurality of polypeptide molecules may comprise one or more different polypeptide molecules. The plurality of polypeptide molecules may comprise a polypeptide complex (e.g., oligomer), or a protein aggregate. The capture unit 410 may comprise a capture molecule 412 and/or a plurality of capture sites, for example, 413a-c. The capture unit 412 may be immobilized on the support 401 by a crosslinker 414. The plurality of capture sites 413a, 413b, or 413c may couple to capture domains 422a-c in polypeptide molecules 421a-c. The polypeptide molecules may comprise a similar capture domain. The polypeptide molecules 421a-c may comprise similar or different amino acid sequences in a region other than the capture domain region. The polypeptide molecules 421a-c may also comprise binding units 424a-c. The plurality of capture sites 413a-c may be configured to couple to different capture domains 422a-c in similar or different polypeptide molecules. This may allow for capturing similar polypeptide molecules suing different capture domains, which may in turn allow identifying potential differences in binding affinity between various capture site and capture domain pairs; or it may be used to identify variations of similar polypeptide molecules of interest (e.g., various mutations or folding differences in similar polypeptide molecules).

The reporter moiety 430 may comprise a plurality of reporter molecules 434a-c, a plurality of recognition units 432a-c, and/or a plurality of detectable labels 438a-c. The reporter moiety 430 may also comprise a plurality of spacers 436a-c, wherein the plurality of reporter molecules 434a-c and the plurality of detectable labels 438a-c may be coupled via the plurality of spacers. The plurality of recognition units may be configured to couple to similar binding units in similar or different polypeptide molecules. For example, polypeptide molecules 421a and 421b may be different from one another but they may comprise similar binding units 424a and 424b. The plurality of recognition units may be configured to couple to different binding units in similar or different polypeptide molecules. In another example, polypeptide molecules 421a and 421c may be different from one another and/or they may also comprise different binding units 424a and 424c. This may allow for labeling different polypeptide molecules. In another example, binding units 424b and 424c may be different binding units in similar polypeptide molecules 421b and 421c. Therefore, recognition units 432b and 432c may be configured to recognize and/or couple to the different binding units 424b and 424c in the similar polypeptide molecules 421b and 421c. This may allow for labeling different variations of similar polypeptide molecules (e.g., various mutations or folding differences in similar polypeptide molecules).

The methods described herein may also be used to measure binding strength of different recognition units and/or binding units for targeting similar or different molecules of interest. The molecules of interest can be polypeptide molecules (e.g., protein subunits in aggregated proteins or protein repeats in protein tandem repeats). The detectable labels 438a, 438b, 438c may each comprise a different detectable signal to allow detecting similar and/or different polypeptide molecules substantially simultaneously. The detectable labels 438a, 438b, 438c may be used in a FRET assay. For example, detecting two or more of polypeptide molecules 42 la-c by detecting the signal from two or more of the detectable labels 438a-c may lead to at least one signal generated by FRET where the FRET signal is different from the signals that can be detected from the each of the detectable labels 438a-c.

In some cases, a method of the disclosure may comprise providing a polypeptide complex comprising a capture unit, a polypeptide molecule, and a reporter moiety. In some cases, the capture unit may be coupled to a support and/or comprise a plurality of capture sites, which bind to a plurality of capture domains of the polypeptide molecule. In some cases, the polypeptide may comprise a plurality of capture domains that bind to a plurality of capture sites of a capture unit. In some cases, the polypeptide may comprise a plurality of binding units that bind to a plurality of recognition unit of a reporter moiety or reporter molecule. In some cases, a reporter moiety may comprise a reporter molecule that comprises a plurality of recognition units that bind to a plurality of binding units of a polypeptide molecule. In some cases, a reporter molecule may further comprise a detectable label that is coupled to the reporter molecule, for example, by a covalent bond.

FIG. 5A illustrates another embodiment of the methods disclosed herein. Method 500a may comprise a support 501, a capture unit 510, a polypeptide complex 520, and/or a reporter moiety 530. The capture unit 510 may comprise a capture molecule 512 and/or a plurality of capture sites 513a-c. A capture unit 510 may be coupled directly to a support 501. A capture unit 510 may be coupled to a support 501 using a crosslinker similar to a construct 410 shown in FIG. 4. The plurality of capture sites 513a-c may be configured to couple to one or more capture domains 522a-c of a plurality of polypeptide molecules 521a-c (e.g., monomers in an oligomer) in a polypeptide complex 520. The plurality of polypeptide molecules 521a-c may also comprise a plurality of binding units 524a-c. The one or more capture domains 522a-c may be similar to one another or may be different. Using one or more similar capture domains may help to increase a strength of binding for more stable capturing. Similar capture domains may exist in similar or different polypeptide molecules in a polypeptide complex. Therefore, the capture unit 510 may capture a polypeptide complex by binding to a plurality of similar or different capture domains in similar or different subunits of that polypeptide complex.

The reporter moiety 530 may comprise a plurality of recognition units 532a-c, a reporter molecule 534, and/or a detectable label 538. The reporter moiety 530 may also comprise a spacer 536, wherein the reporter molecule 534 and the detectable label 538 may be coupled to one another via the spacer 536. The plurality of recognition units 532a-c may be configured to couple to the plurality of binding units 524a-c in the polypeptide complex 520. The one or more binding units 524a-c may be similar to one another or different. A plurality of recognition units 532a-c may be used to bind to similar binding units 524a-c to achieve a higher affinity of binding; or in some case it may be used for specific binding of a reporter moiety to a polypeptide complex of interest with a predefined number of subunits. Using a plurality of recognition units 532a-c to bind to binding units 524a-c, where the binding units are different from one another, may allow to bind a reporter moiety to a polypeptide complex of interest with specificity.

FIG. 5B illustrates another embodiment of the methods disclosed herein. Method 500b may comprise a support 501, a capture unit 560, a polypeptide complex 570 (e.g., polypeptide repeat), and/or a reporter moiety 580. The polypeptide complex may be a protein repeat. The capture unit 560 may comprise a capture molecule 562 and/or a plurality of capture sites 563a-d. The plurality of capture sites 563a-d may be configured to couple to one or more capture domains 572a-d in the polypeptide complex 570 (e.g., polypeptide tandem repeat). Each of the plurality of capture sites 563a-d may be configured to couple to one capture domain in the plurality of capture domains 572a-d in the protein complex 570 (e.g., polypeptide tandem repeat). The capture unit 560 may use a plurality of capture sites 563a-d to bind to the polypeptide complex 570 with higher specificity. For example, a polypeptide complex with a structure or a fold not matching the plurality of capture sites may not be captured, allowing for higher specificity. The polypeptide complex 570 may comprise a plurality of binding units 574a-d. The reporter moiety 580 may comprise a plurality of recognition units 582a-d, a reporter molecule 584, or a detectable label 588. The reporter moiety 580 may also comprise a spacer 586, wherein the reporter molecule 584 and the detectable label 588 may be coupled via the spacer 586. The plurality of recognition units 582a-d may be configured to couple to one or more binding units 574a-d in the polypeptide complex 570 (e.g., polypeptide tandem repeat). Each of the plurality of recognition units 582a-d may be configured to couple to one binding unit of the plurality of binding units 574a-d in the polypeptide complex 570 (e.g., polypeptide repeat).

In some cases, a method of the disclosure may comprise providing a polypeptide complex comprising a substrate, a plurality of capture units, a plurality of polypeptide molecules, and a plurality of reporter moieties. In some cases, the substrate can be bound directly to a solid support or bound to the solid support via a covalently bonded crosslinker. In some cases, the substrate may comprise a plurality of capture units. In some cases, the plurality of capture units can be bound to the substrate directly or by a covalently bonded crosslinker. In some embodiments, the substrate is a PDMS imprint with functionalized capture antibodies. In some cases, each of the plurality of capture units may comprise one or more capture sites, which bind to one or more capture domains of one or more polypeptide molecules. In some cases, the polypeptide molecules may comprise one or more capture domains that bind to one or more capture sites of a capture unit. In some cases, the polypeptides may comprise one or more binding units that bind to one or more recognition units of a reporter moiety. In some cases, a reporter moiety may comprise one or more reporter molecules that comprise one or more recognition units that bind to a polypeptide molecule. In some cases, the one or more reporter molecules may further comprise one or more detectable labels that are coupled to the one or more reporter molecules, for example, by a covalent bond.

FIG. 6 illustrates another embodiment of the methods described herein, where a plurality of different and/or similar polypeptide molecules and/or polypeptide complexes may be captured and/or labeled. The polypeptide molecules and/or polypeptide complexes may be from one or more samples. In some cases, method 600 may be used to capture a plurality of molecules of interest (e.g., polypeptide molecules or polypeptide complexes) in a heterogenous sample (e.g., blood, CSF, plasma, serum, urine, saliva, mucosal excretions, sputum, stool or tears). Method 600 may comprise a support 601, a substrate 602, a plurality of capture units 610a-c, a plurality of polypeptide molecules or polypeptide complexes 620a-d, or a plurality of reporter moieties 630a-c. The substrate 602 may be coupled to a support 601 using a crosslinker 614. The plurality of capture units 610a-c may capture a plurality of similar or different polypeptide molecules and/or complexes. In some cases, similar capture units such as capture unit 610a may be used to capture a polypeptide molecule 620a, a polypeptide complex 620b, or a polypeptide complex 620d. Capturing larger polypeptide complexes such as polypeptide complex 620c may require a higher binding strength (e.g., binding affinity) than a capture unit with a single capture site such as capture unit 610a may be able to provide. In some cases, a capture unit with one or more capture sites such as capture unit 610b or 610c may be used for a higher binding strength to capture a larger molecule of interest such as 620b-d. In some cases, a capture unit with one or more capture sites may also be used for higher specificity of binding. For example, capture unit 610c may be configured to bind to a polypeptide complex (e.g., a protein tandem repeat) with a predefined fold or structure.

The captured molecule of interest may be labeled using one or more of the plurality of reporter moieties 630a-c. Each reporter moiety may carry at least one detectable label. The detectable labels 635a-c may produce detectible signals that may be different from one another or may be similar. In some cases, two or more reporter moieties may bind to a molecule of interest (e.g., a polypeptide complex); the detectable labels bound to the two or more reporter moieties may then be close enough to produce a detectable signal via FRET. The different signals detected from the plurality of detectable labels may be used to identify and/or distinguish the molecules of interest. The number of subunits in captured and labeled molecules (e.g., polypeptide molecules or polypeptide complexes) may be quantified, for example, by rendering the detectable labels undetectable as described herein.

The detectable label may be configured to emit a signal upon excitation using an energy source (e.g., via a laser). The signal can be a detectable signal. For example, the signal can be an optical signal, such as a fluorescent or phosphorescent signal. The detectable label may comprise a dye. The detectable label may produce an electrical signal, a radioactive signal or a chemical signal. The reporter moiety may be coupled to a spacer. The spacer may adjoin a reporter moiety and the detectable label.

The methods described herein further comprise detecting one or more signals from the polypeptide complex, which one or more signals correspond to the plurality of detectable labels.

To detect one or more signals from the polypeptide complex at least one detectable label of the reporter moiety coupled to at least a polypeptide molecule in a polypeptide complex is excited using an excitation energy source (e.g., light or a laser). The amount of the detectable signals from the detectable labels excited using an excitation energy source can be used to quantify an amount of the polypeptide molecules or polypeptide complex. In some cases, a reporter moiety may comprise one or more of recognition units that may couple to one or more of polypeptide molecules in a polypeptide complex. The reporter moiety may further comprise one or more detectable labels per recognition unit. Therefore, the one or more signals detected from the detectable labels excited using an excitation energy source can be used to quantify an amount of the one or more of polypeptide molecules in the polypeptide complex.

The methods described herein also comprise subjecting the one or more of detectable labels to conditions sufficient to render at most a subset of the one or more of detectable labels undetectable. In some embodiments, the conditions sufficient to render at most a subset of the one or more of detectable labels undetectable includes photobleaching. In some embodiments, the conditions sufficient to render at most a subset of the one or more of detectable labels undetectable includes step wise photobleaching. In some embodiments, the conditions sufficient to render at most a subset of the one or more of detectable labels undetectable includes dye quenching. In some embodiments, the conditions sufficient to render at most a subset of the one or more of detectable labels undetectable includes enzymatic cleavage of the detectable labels. The detectable label may be physically detached or uncoupled from the reporter moiety via photocleaving. The detectible label may be subjected to a first energy source (e.g., light or laser) to cause photobleaching in the detectable label. The detectable label may be rendered undetectable upon excitation using a second energy source followed by detection of detectable signal from reporter moieties. The first energy source may provide an amount of energy larger than the second energy sources. The first and the second energy sources may illuminate the field of view (e.g., microscope imaging) with a laser that can lead to photobleaching. The first energy source may render at least a subset of the one or more of detectable labels undetectable. A change in the intensity of the detectable signals from the one or more of detectable labels can be associated with the number of polypeptide molecules present in a polypeptide complex. The first energy source and the second energy source may be the same. The first energy source and the second energy source may be different. Subsequent photobleaching followed by a detecting of a detectable signal may be performed. Photobleaching and/or detecting process may be repeated until substantially all of the detectable labels may be rendered undetectable. Photobleaching and/or detecting process may be repeated until at least about 50%, 60%, 70%, 80%, 90%, 92%, 94%, 96%, 98% of the one or more of detectable labels may be rendered undetectable. Photobleaching and/or detecting process may be repeated until at least about 50% of the one or more of detectable labels may be rendered undetectable. Photobleaching and/or detecting process may be repeated until at least about 75% of the one or more of detectable labels may be rendered undetectable. Photobleaching and/or detecting process may be repeated until at least about 80% of the one or more of detectable labels may be rendered undetectable. Photobleaching and/or detecting process may be repeated until at least about 90% of the one or more of detectable labels may be rendered undetectable. Photobleaching and/or detecting process may be repeated until at least about 99% of the one or more of detectable labels may be rendered undetectable.

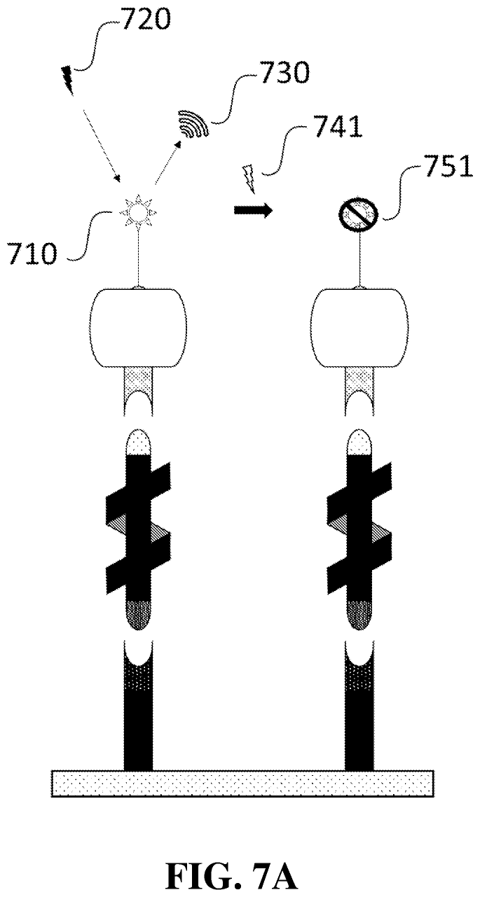

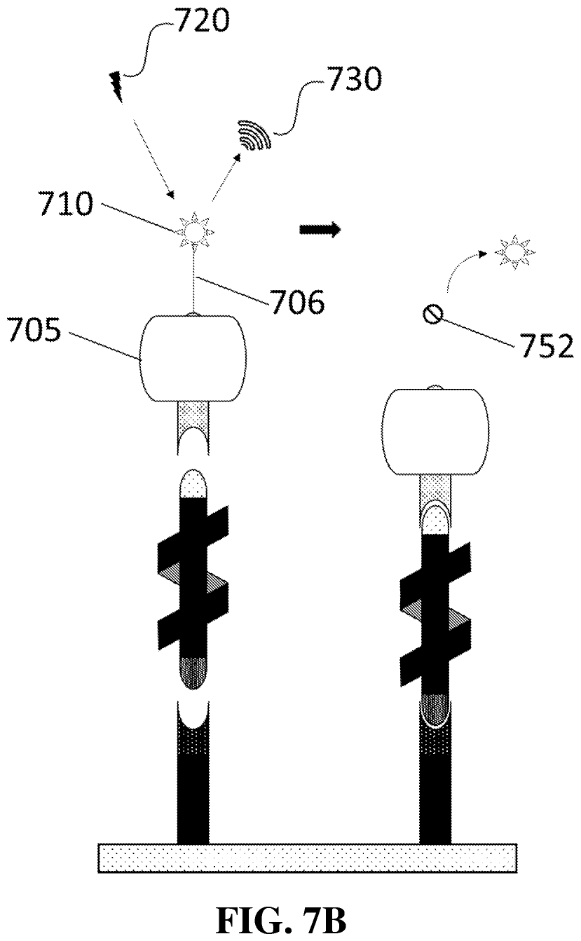

FIG. 7A. illustrates an example of photobleaching according to an embodiment of the methods disclosed herein. A detectable label 710 may be excited using an excitation energy source 720. A signal 730 may be detected from the excited detectable label 710. The excitation energy source 720 may be a laser. The detectable label may be a molecule comprising a fluorophore dye. The signal 730 may be a fluorescent signal. An energy source 741 may be used to render the detectable label 710 becoming an undetectable label 751.

In some cases, the detectable label may be uncoupled from the reporter moiety by cleaving a detectable label from a reporter moiety. The detectable label may be coupled to a reporter moiety by a cleavable linker (e.g., spacer). The cleavable linker may be cleavable by an enzyme. The cleavable linker may be a chemically cleavable linker. The cleavable linker may be a photocleavable linker. The cleavable linker may be capable of being cleaved by a change in pH. Non-limiting examples of cleavage conditions to split a cleavable linker may include: enzymes, nucleophilic or basic reagents, reducing agents, photo-irradiation, electrophilic or acidic reagents, organometallic or metal reagents, or oxidizing reagents.

FIG. 7B. illustrates an example of processing a polypeptide according to an embodiment of the methods disclosed herein. A detectable label 710 can be excited using an excitation energy source 720. A signal 730 may be detected from the excited detectable label 710. The excitation energy source 720 can be a laser. The detectable label can be a molecule comprising a fluorophore dye. The signal 730 can be a fluorescent signal. The detectable label 710 may be rendered undetectable by uncoupling the detectable label 710 from the reporter molecule 705. The detectable label 710 may be uncoupled from the reporter molecule 705 via a cleaving system. The cleaving system may cleave the spacer 706 (e.g., a crosslinker) to turn the detectable label 710 into an undetectable label 752. The cleaving process may comprise photocleaving reaction. Photocleavage reaction may comprise, for example, an ion pair from an excited ester and a corresponding alcohol may be split by applying energy (e.g., light or laser). The cleaving process may comprise chemical cleaving reaction using, for example, an enzyme (e.g., restriction enzyme).

In some cases, a polypeptide molecule (e.g., monomer) in a protein aggregate (e.g., oligomer) can be coupled to at least one reporter moiety. On the other hand, a reporter moiety may carry or be coupled to at least one detectable label. Therefore, the number of polypeptide molecules in a sample (e.g., monomer counts in a sample) and/or in a polypeptide complex (e.g., monomer counts in an oligomer indicating an oligomer size) can be quantified by quantifying the signals from the detectable labels. In some other cases, a polypeptide complex (e.g., an oligomer) can be coupled to at least one reporter moiety and the reporter moiety may be coupled to or carry at least one detectable label. Therefore, the number of oligomers present in a sample can be quantified (e.g., oligomer counts) by quantifying the signals from the detectable labels.

In some cases, the intensity of the signal can be used to quantify the detectable signal from a polypeptide complex. In some cases, a signal can be quantified by counting a number of repeating cycles, where a signal is rendered undetectable, sufficient to render substantially all the detectable labels undetectable. In some cases, the intensity of the detected signal can be correlated with a size of the polypeptide complex (e.g., number of polypeptides in an oligomer). In some cases, the intensity of the detected signal can be used to calculate the number of polypeptide molecules that may be present in a polypeptide complex or in a sample. The polypeptide molecules may be present in a sample either individually (e.g., single monomers) or in polypeptide complexes (e.g., oligomers with two or more monomers). For example, a heterogenous sample may include single monomers, as well as one or more of oligomers comprising different number of monomers (e.g., an oligomer with two, three, four, or five subunits also known as a dimer, trimer, tetramer, pentamer, respectively). The number of polypeptides in a sample may include the number of single monomers, dimers, trimers, tetramers, pentamers, and/or larger polypeptide complexes in the heterogenous sample.

A plurality of parameters can be correlated or measured directly from the detected signals. The plurality of parameters may comprise monomer counts, oligomer counts, oligomer size (e.g., oligomer with two, three, four, five, or more monomers), a frequency of polypeptide molecule counts, a distribution of the frequency of monomer counts, a mode of the distribution of the frequency of monomer counts and/or other parameters. A mode of the distribution of the frequency of monomer counts may in turn comprise a shift in the distribution. The shift in the distribution of the frequency of monomer counts can also be a parameter that may be indirectly correlated or measured from the detected signals. The plurality of parameters directly measured or correlated from the detected signals can be used as quantitative measurement with diagnostic potential. The plurality of parameters can be correlated or associated with a disease or disorder comprising clinical symptoms of the disease or disorder, stage of the disease or disorder, progression of the disease or disorder, and/or treatment choices for the disease or disorder.

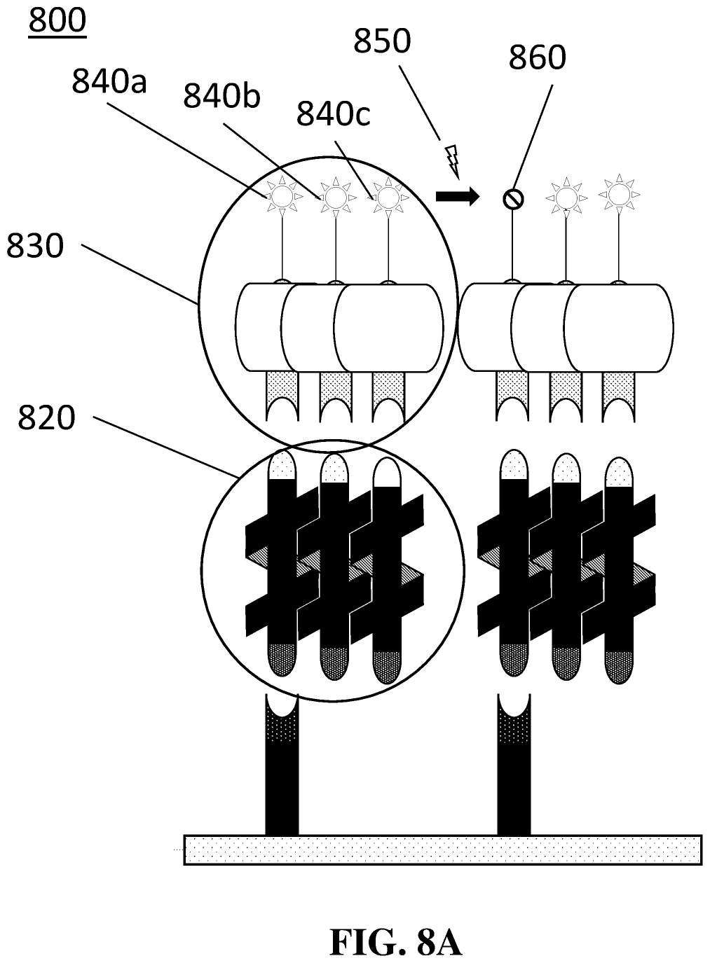

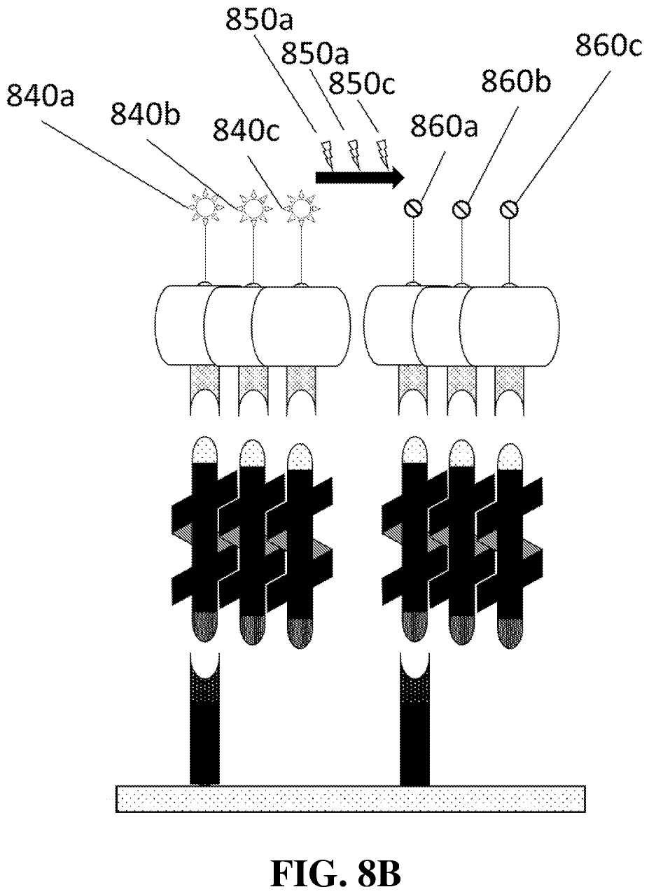

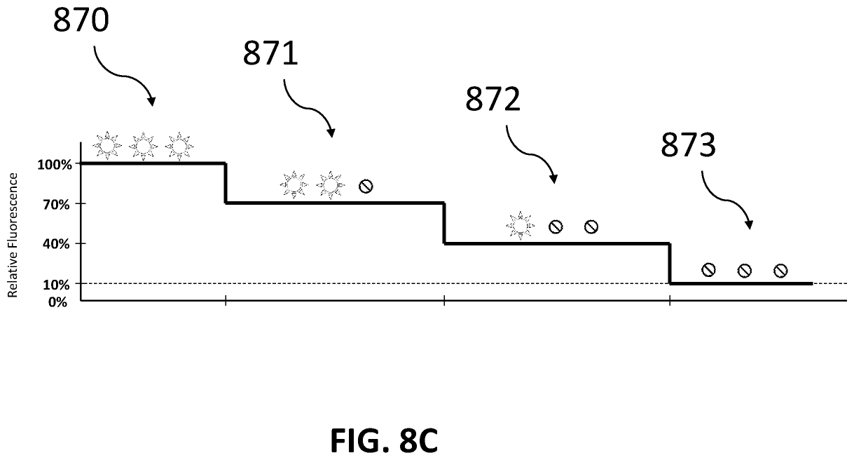

FIGS. 8A-C illustrate an example of quantifying a signal from detectable labels of a reporter moiety coupled to a polypeptide complex. In method 800 shown in FIG. 8A, a polypeptide complex 820 may comprise a plurality of polypeptide molecules. One or more reporter moieties 830 may then be coupled to one or more of the plurality of polypeptide molecules in the polypeptide complex 820. Each reporter moiety may comprise a detectable label. The plurality of detectable labels 840a-c may generate a signal and may be detected using the signal. Next, an energy source 850 can be provided to render at most one of the pluralities of detectable labels 840a-c undetectable 860. This process may include photobleaching. In some cases, the energy source 850 may be sufficient to render at least about 5%, 10%, 20%, 30%, 40%, 50%, 60%, 70%, 80%, 90%, or more of the plurality of detectable labels undetectable. The energy source 850 may be sufficient to render at most about 90%, 80%, 70%, 60%, 50%, 40%, 30%, 20%, 10%, or less of the plurality of detectable labels undetectable. Detection and/or photobleaching steps may be repeated until essentially all the detectable labels are undetectable.

For example, FIG. 8B illustrates embodiments for rendering detectable labels undetectable. A plurality of reporter moieties may be coupled to a polypeptide complex. A first energy source 850a may be applied to render a first detectable label 840a from the plurality of labels undetectable 860a. Then, a second energy source 850b may be applied to render a second detectable label 840b from the plurality of labels undetectable 860b. A third energy source 850c may be applied to render a third detectable label 840c from the plurality of labels undetectable 860c. As shown in FIG. 8B, not intended to be limiting of any embodiments of the methods described herein, repeating a signal removing step (e.g., quenching or photobleaching) three times may be required to render the detectable labels 840a-c undetectable. This may be correlated with the number of polypeptide molecules in the polypeptide complex that is being detected.

FIG. 8C illustrates quantification of the aforementioned repeating cycles of detection and/or photobleaching. Initial detection of the one or more signals from the polypeptide complex 820 may be considered maximum or 100% relative fluorescence level detected 870. After providing the first energy source to render a subset of the detectable labels undetectable (e.g., by photo bleach), the detected signal may be reduced to a second relative fluorescence level 871. Next, a second energy source may be applied to render a subset of the remaining detectable labels undetectable. The detected signal may be reduced to a third relative fluorescence level 872. The cycle of applying an energy source to partially photo bleach a subset of remaining detectable labels can be continued until essentially no signal can be detected from the polypeptide complex. For example, as shown in FIG. 8B, the third energy source can also be applied. At this point, as shown in FIG. 8C, the detected signal can be reduced to a relative fluorescence level equivalent to a baseline relative fluorescence level 873. The baseline can be at least about 0%, 1%, 2%, 3%, 4%, 5%, 6%, 7%, 8%, 9%, 10%, 12%, 14%, 16%, 18%, 20%, 25%, 30%, or more fluorescence signal relative to the maximum or 100% signal detected prior to a signal removing step (e.g., quenching or photobleaching), a signal is removed from detectable label coupled to a polypeptide complex (e.g., photobleaching or photocleaving).

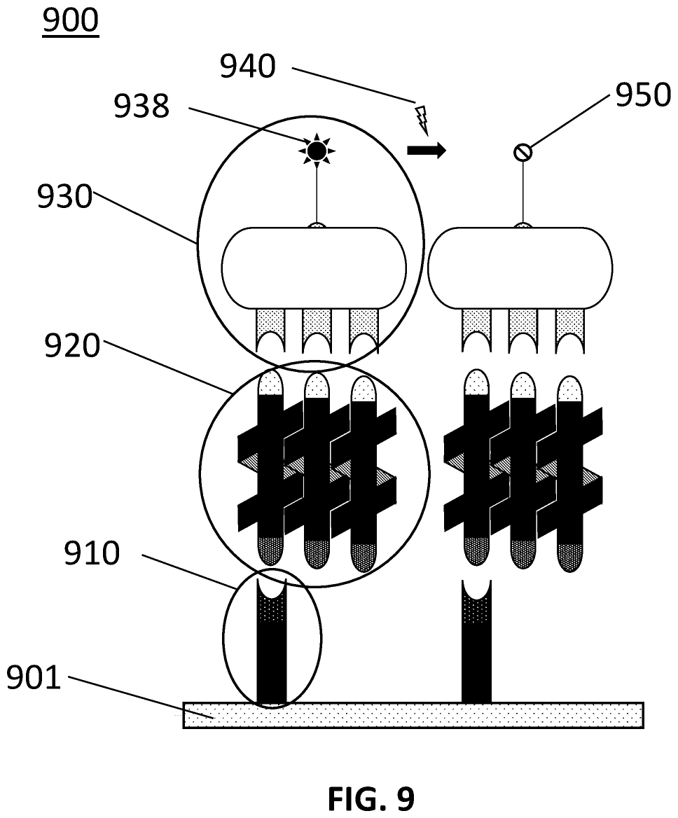

FIG. 9 illustrates another embodiment of detecting polypeptide complexes. A method 900 may be used to detect a polypeptide complex 920 that may comprise one or more polypeptide molecules. The polypeptide complex may be coupled to capture unit 910, where the capture unit 910 is immobilized on a support 901 (e.g., by using a crosslinker). The method may further comprise a reporter moiety 930 comprising one or more recognition units. The one or more recognition units may be configured to bind to the one or more polypeptide molecules in the complex 920. The recognition units in the reporter moiety 930 may be configured to selectively bind to the polypeptide complex 920. The reporter moiety may also comprise a reporter moiety 938. The polypeptide complex 920 may be detected by detecting a signal emitted from the detectable label 938. The detectable label 938 may be rendered undetectable 950 using an energy source 940. This may allow detecting polypeptide complex 920 in a sample with other labeled molecules. For example, a polypeptide complex may comprise a plurality of polypeptide molecules similar to one or more polypeptide molecules in the complex 920. The larger polypeptide complex may be 2 times, 3 times, 4 times or more larger than the complex 920 (e.g., a protein aggregate with similar subunits or a protein tandem repeat with similar repeating subunits). Subsequently, the larger polypeptide complex may be labeled with two or more reporter moieties similar to the reporter moiety 930. By providing an energy source sufficient to render one detectable label coupled to a polypeptide complex undetectable the polypeptide complex 920 carrying the label 938 may lose the signal 950 while a larger polypeptide complex with two or more detectable labels may be detectable.

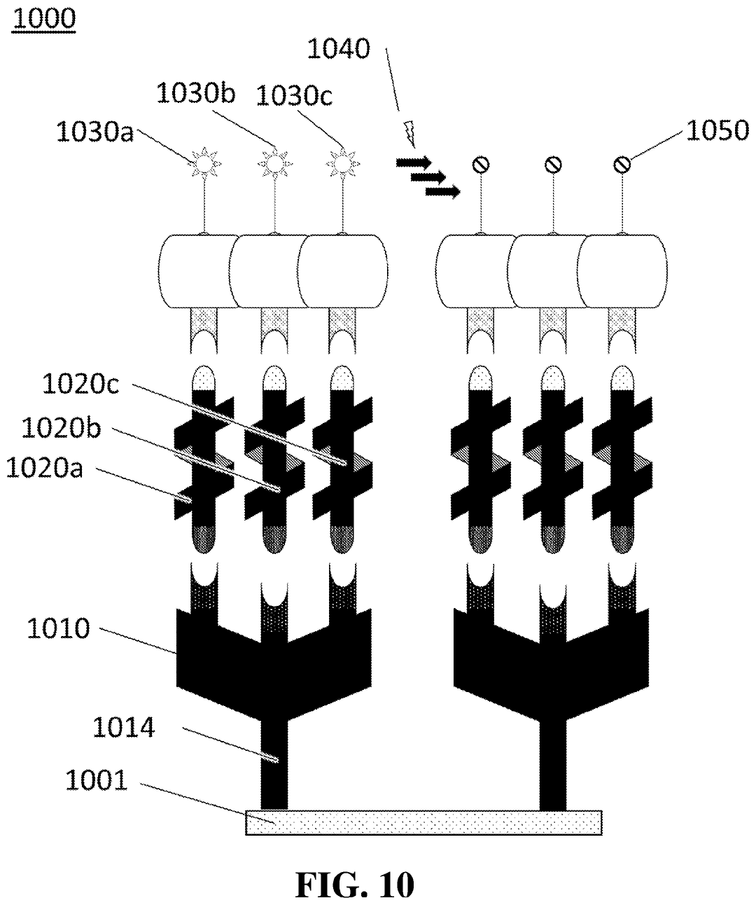

FIG. 10 illustrates an embodiment of a method for detecting polypeptide molecules. Method 1000 may comprise detecting one or more polypeptide molecules 1020a-c. The one or more polypeptide molecules 1020a-c may be captured using a capture unit 1010 that may be configured to capture a plurality of polypeptide molecules. The capture unit may be coupled to a support 1001 via a crosslinker 1014. The capture unit 1010 may be similar to capture unit 410. A reporter moiety carrying a detectable label (e.g., detectable labels 1030a-c) may be coupled to each of the one or more polypeptide molecules 1020a-c. The detectable labels 1030a-c may be subjected to a photobleaching process 1040. The photobleaching process 1040 may comprise one or more steps of providing an energy source (e.g., light or laser). Each step of the photobleaching 1040 may render at least one detectable label undetectable (e.g., an undetectable label 1050). A change in the detected signal from the detectable labels can be correlated with the number of polypeptide molecules captures and/or labeled. In some cases, a number of repeating steps in the photobleaching process 1040 to render substantially all the detectable labels undetectable may be correlated with the number of polypeptide molecules captured and/or labeled.

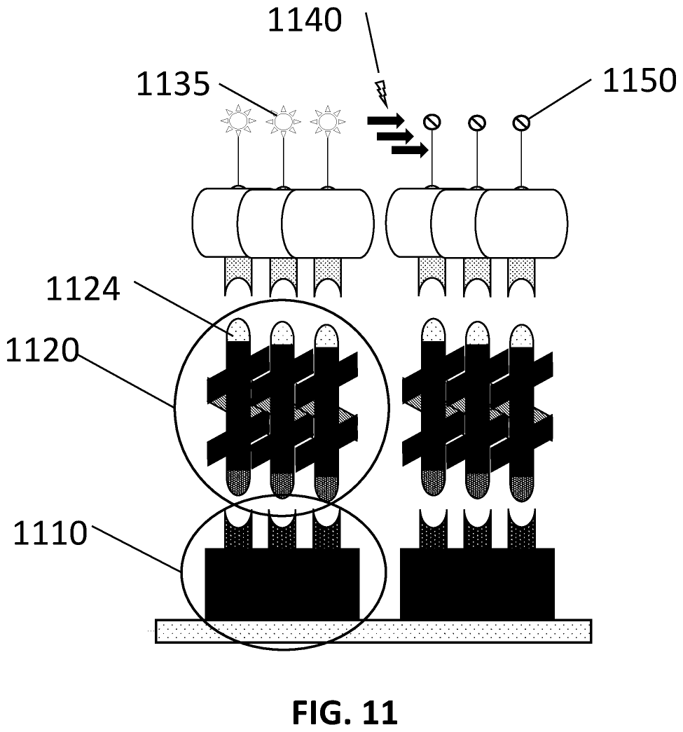

FIG. 11 shows another embodiment of detecting a polypeptide complex. A polypeptide complex 1120 may be captured by a capture unit 1110. The capture unit may comprise a plurality of capture sites that may allow the capture unit to couple to a plurality of polypeptide molecules in the polypeptide complex 1120. The polypeptide complex may comprise binding units 1124. The capture unit 1110 may specifically capture a polypeptide complex with a predetermined number of polypeptide molecules (e.g., oligomer with three, four, or more subunits). The polypeptide complex (e.g., an oligomer) may comprise at least 2, 3, 4, 5, 6, 7, 8, 9, 10, 12, 13, 14, 15, 16, 17, 18, 19, 20, 21, 22, 23, 24, 25, 30, 40, 50, 100 or more polypeptide molecules (e.g. tandem repeats or monomer subunits). Next, a plurality of reporter moieties carrying detectable labels (e.g., detectable label 1135) may be coupled to the polypeptide complex 1120. The detectable labels may be first detected and then be subjected to an energy source 1140 to render at least one detectable label (e.g., detectable label 1135) undetectable (e.g., undetectable label 1150). The detection and/or photobleaching may be repeated until substantially no signal can be detected from the polypeptide complex. A number of cycles required to render substantially all the detectable labels undetectable can be quantified and/or may be correlated with the number of polypeptide molecules in the polypeptide complex 1120.

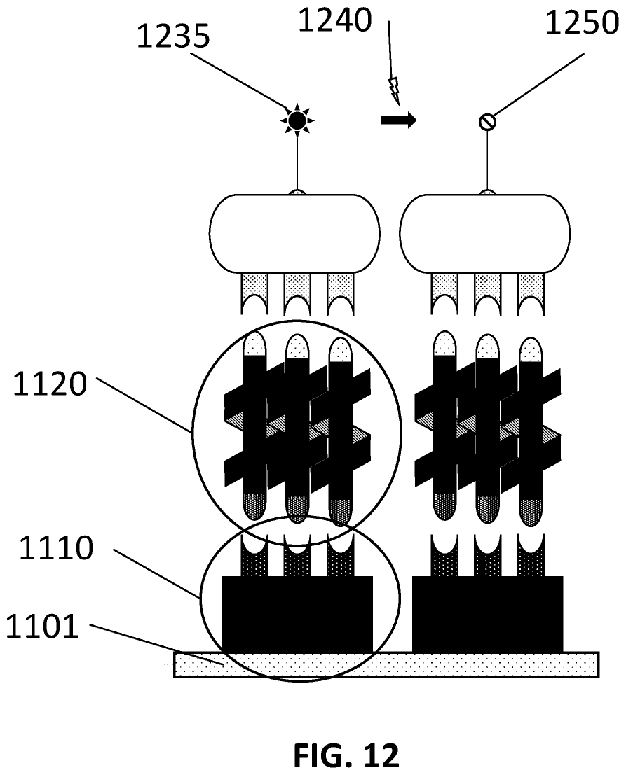

A reporter moiety carrying one detectable label 1235 may be used to label the polypeptide complex 1120 as illustrated in FIG. 12. The reporter moiety may be configured to selectively couple to polypeptide complex 1120. Therefore, detecting a signal from the detectable label 1235 may be used to detect the polypeptide complex 1120 in a sample. The detectable label 1235 may be rendered undetectable 1250 in a single photobleaching step 1240.

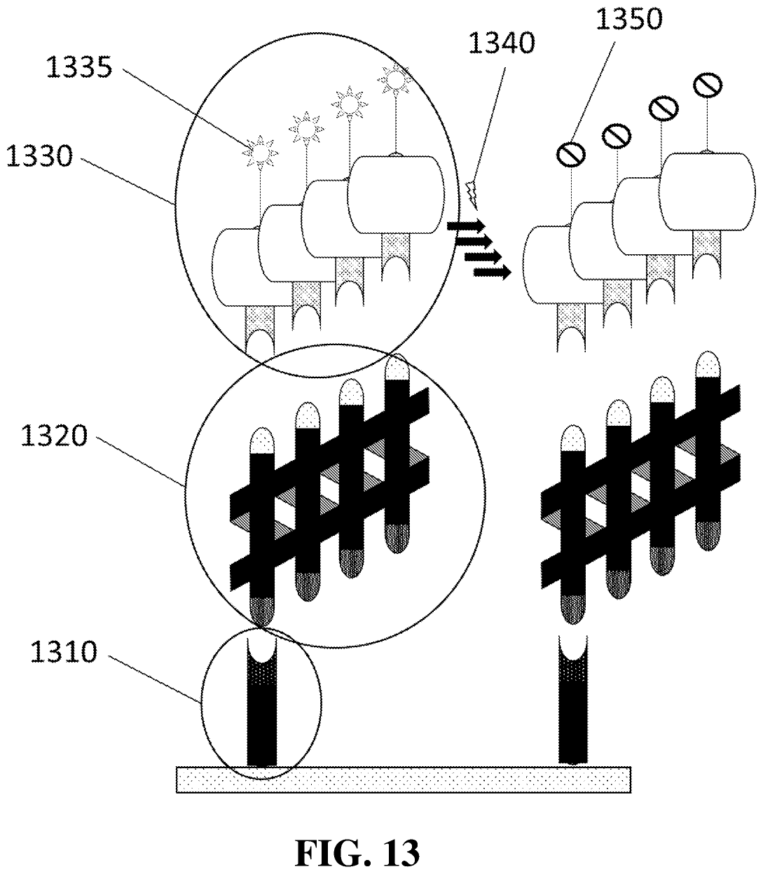

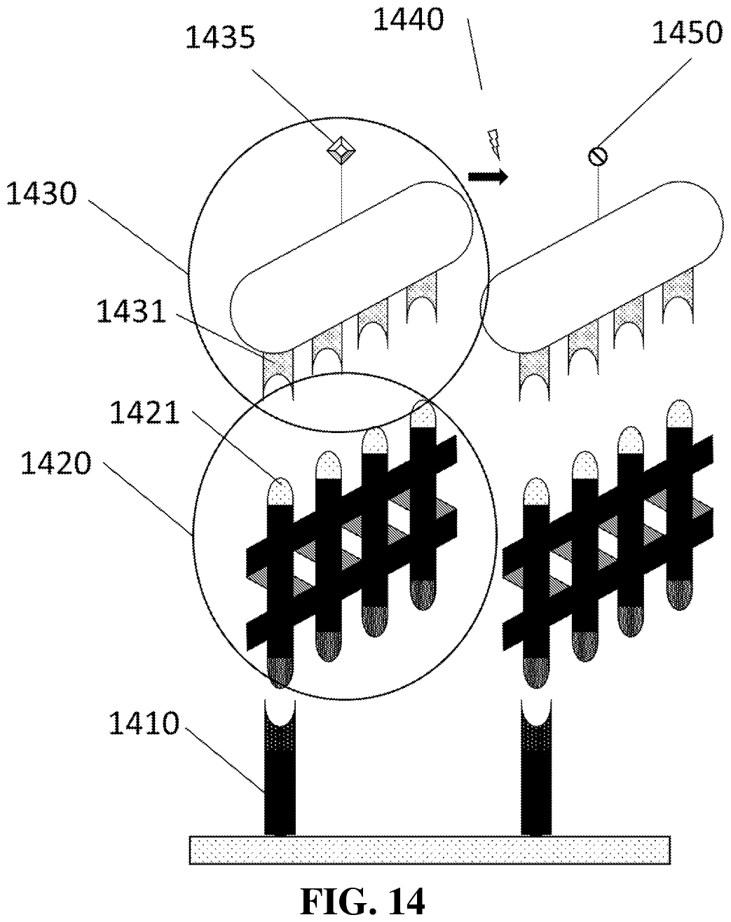

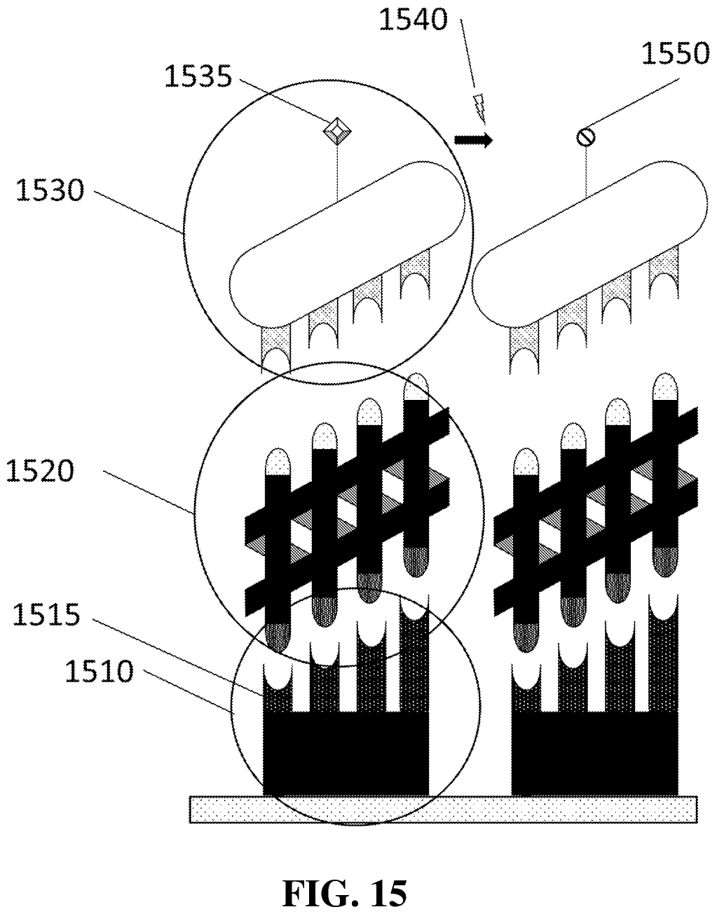

FIG. 13, FIG. 14, and FIG. 15 schematically illustrate examples of detecting a polypeptide complex, where the polypeptide complex may be an array of protein tandem repeats 1320. As shown in FIG. 13, the polypeptide complex 1320 (e.g., array of protein tandem repeats) may comprise a plurality of polypeptide molecules (e.g., units of protein tandem repeats in an array). The polypeptide complex 1320 may be captured via a capture unit 1310. The capture unit 1310 may capture the polypeptide complex 1320 by binding to at least one polypeptide molecule in the polypeptide complex 1320. The method may further comprise a reporter moiety 1330 comprising one or more recognition units. The one or more recognition units can also comprise a detectable label 1335. The polypeptide complex 1320 can be detected by detecting a signal emitted from the detectable label 1335. The detectable label 1335 may be rendered undetectable 1350 using an energy source 1340.

As shown in FIG. 14, a reporter moiety 1430 with a plurality of recognition units (e.g., recognition unit 1431) may be used to label the polypeptide complex 1420; where the reporter moiety 1430 may be coupled to a plurality of polypeptide molecules in the polypeptide complex 1420 at once. This may allow for stronger binding between the reporter moiety 1431 and the polypeptide complex 1420. It may also allow for selective labeling of the polypeptide complex 1420. For example, the reporter moiety 1430 may not couple to a polypeptide complex that may have a protein fold and/or a protein structure that is different from the polypeptide complex 1420 in spite of a similarity in their polypeptide molecules. In some cases, the reporter moiety 1430 may be used to label a polypeptide complex with more polypeptide molecules than 1420 (e.g., different arrays of similar protein tandem subunits). Each of the individual reporter moieties in the plurality of reporter moieties 1330 may carry a detectable label (e.g., detectable label 1435), labeling the polypeptide complex 1320 with a plurality of detectable labels. A source of energy 1440 may be applied to render at least one detectable label undetectable (e.g., undetectable label 1450). This process may be repeated more than once. The number of times this process may be repeated to render substantially all the detectable labels undetectable may be correlated with a number of polypeptide molecules in the polypeptide complex 1420. The reporter moiety 1430 may carry a detectable signal 1435 and may be rendered undetectable 1450 using an energy source 1440.

As shown in FIG. 15, the polypeptide complex 1520 may be captured via a capture unit 1510. The capture unit 1510 may comprise a plurality of capture sites (e.g., capture site 1515) to capture a plurality of polypeptide molecules in the polypeptide complex 1520. This may allow stronger binding affinity between the capture unit and the polypeptide complex and/or may promote selective capturing of the polypeptide complex 1520 in a heterogenous sample. A heterogenous sample may comprise two or more different polypeptide complexes. The capture units 1510 may comprise an antibody. To label and/or detect the polypeptide complex 1520, one or more reporter moieties can be coupled to one or more the polypeptide molecules in the polypeptide complex 1520. Individual reporter moieties in a plurality of reporter moieties 1530 may be coupled to each of the polypeptide molecules in the polypeptide complex 1520.

### Detecting a Disease or Disorder

Provided herein are methods for detecting a disease or disorder in a subject. The methods may comprise providing a polypeptide complex from a subject comprising a plurality of polypeptide molecules, wherein the polypeptide complex may be coupled to a capture unit immobilized to a support. A plurality of reporter moieties comprising a plurality of detectable labels may then be coupled to the polypeptide complex. Next, one or more signals corresponding to the plurality of detectable labels can be detected from the polypeptide complex. The plurality of detectable labels may be subjected to conditions sufficient to render at most a subset of the plurality of detectable labels undetectable. The disease or disorder in the subject can be detected based at least in part on the one or more signals, corresponding to the plurality of detectable labels, that may be detected from the polypeptide complex.

In some embodiments, a label eliminating step comprising subjecting the plurality of detectable labels to conditions sufficient to render at most a subset of the plurality of detectable labels undetectable can be repeated. The label eliminating step may reduce an intensity of the detectable labels in a plurality of reporter moieties that may be coupled to the polypeptide complex. A number of polypeptide molecules in a polypeptide complex (e.g., an oligomer) may be determined by repeating the eliminating step until substantially all labels in a reporter moiety that may be coupled to a polypeptide complex are rendered undetectable. For example, number of decreases in intensity of fluorescent signals that may be detected at a location on a support (e.g., fluorescent imaging slide) representing a polypeptide complex may be used to count a number of polypeptide molecules in the polypeptide complex. A distribution of the number of polypeptide molecules in the polypeptide complex in a sample from a subject may be compared with a control or reference sample. The control or reference sample may be from a healthy individual or healthy tissue of the same subject. A difference in the distribution of the number of polypeptide molecules in the sample from the subject compared to the control or reference sample may show an abnormality in protein folding and/or oligomerization of the polypeptide molecules.

Not meant to be limiting, methods as described herein comprising measuring oligomeric counts may be used to establish whether an individual has a neurodegenerative disease (e.g., Alzheimer's Disease). The methods as described herein may be used to measure oligomeric counts of alpha-synuclein in complexes or aggregates in an individual's biological samples. In some cases, a method identifies alpha-synuclein, beta-synuclein, gamma-synuclein, or a combination thereof. In some cases, a method identifies a ratio of alpha- and beta-synuclein levels, alpha- and gamma-synuclein levels, or beta- and gamma-synuclein levels. In some instances, the methods as described herein may be used to determine whether the individual has a disease where the aggregation of the alpha-synuclein is predictive of the disease. For instance, aggregation of the alpha-synuclein is predictive of formation of synucleinopathies such as Parkinson's disease (PD), dementia with Lewy bodies (DLB), and/or multiple system atrophy (MSA). In some instances, the methods as described herein may be used to determine the stage of the disease. In some instances, the methods as described herein may be used to determine if the individual has an early-onset form of the aforementioned diseases.

### Biomarkers

### Alpha-Synuclein

Provided herein are clinical assays for measuring oligomeric counts of alpha-synuclein in complexes or aggregates in patient's biological samples. The aggregation of the alpha-synuclein may be predictive of the formation of synucleinopathies such as Parkinson's disease (PD), dementia with Lewy bodies (DLB), and/or multiple system atrophy (MSA). Parkinson's Disease. The assays may use a single molecule sandwich ELISA method comprising (a) measuring the population of oligomers in a large heterogeneous mixture of single proteins (b) providing a highly linear response. In some cases, a dual-mode single-molecule fluorescence assay may be used to measure oligomeric counts of alpha-synuclein in complexes or aggregates in a biological sample. In some cases, a dual-mode single-molecule fluorescence assay may acquire two parallel independent measures of oligomeric count numbers using: 1) the number of bound detectable labels, which may be derived from the fluorescence intensity of the target-bound and labeled probes; and 2) the direct physical length of the alpha-synuclein oligomers. See, for example, Cannon B, Pan C, Chen L, Hadd A G, Russell R (2013) A dual-mode single-molecule fluorescence assay for the detection of expanded CGG repeats in Fragile X syndrome. Mol Biotechnol 53: 19-28. pmid:22311273, which is incorporated by reference. The assays described herein may be generalized to work on other protein aggregates such as Tau protein for detecting other disorders and/or diseases associated with protein misfolding such as Taupathies.

Other polypeptide complexes may be detected using the methods described herein that may be used as biomarkers for various diseases or disorders. The other polypeptide complexes may include amyloid protein, an amyloid fibril, amyloid beta, amyloid precursor protein, tau protein, microtubule-associated protein tau, alpha synuclein, immunoglobulin, islet amyloid polypeptide, huntingtin protein, FMRP, a polyglutamine repeat protein, a dipeptide repeat protein, TDP-43, matrin-3, or a prion.

### Computer Systems

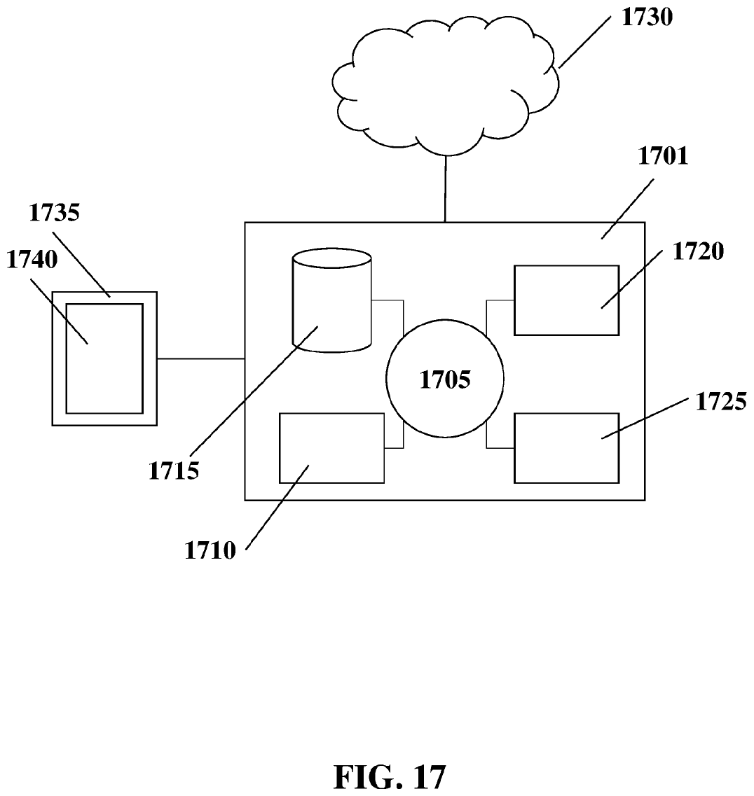

The present disclosure provides computer systems that are programmed to implement methods of the disclosure. FIG. 17 shows a computer system 1701 that is programmed or otherwise configured to implement methods or parts of methods provided herein. The computer system 1701 may regulate various aspects of the present disclosure, such as, for example, controlling an energy source to excite one or more detectable labels, detecting and/or quantifying one or more signals from detectable labels, controlling an energy source to render one or more detectable labels undetectable, measuring a change in detectable signals, correlating a change in the detectable signal with a quantity of polypeptide molecules and/or oligomers, controlling repeating excitation, photobleaching or photocleaving, and/or signal detection cycles. The computer system 1701 may be an electronic device of a user or a computer system that is remotely located with respect to the electronic device. The electronic device may be a mobile electronic device.

The computer system 1701 includes a central processing unit (CPU, also “processor” and/or “computer processor” herein) 1705, which may be a single core or multi core processor, or a plurality of processors for parallel processing. The computer system 1701 also includes memory or memory location 1710 (e.g., random-access memory, read-only memory, flash memory), electronic storage unit 1715 (e.g., hard disk), communication interface 1720 (e.g., network adapter) for communicating with one or more other systems, and/or peripheral devices 1725, such as cache, other memory, data storage and/or electronic display adapters. The memory 1710, storage unit 1715, interface 1720 and/or peripheral devices 1725 are in communication with the CPU 1705 through a communication bus (solid lines), such as a motherboard. The storage unit 1715 may be a data storage unit (or data repository) for storing data. The computer system 1701 may be operatively coupled to a computer network (“network”) 1730 with the aid of the communication interface 1720. The network 1730 may be the Internet, an internet and/or extranet, or an intranet and/or extranet that is in communication with the Internet. The network 1730 in some cases is a telecommunication and/or data network. The network 1730 may include one or more computer servers, which may enable distributed computing, such as cloud computing. The network 1730, in some cases with the aid of the computer system 1701, may implement a peer-to-peer network, which may enable devices coupled to the computer system 1701 to behave as a client or a server.

The CPU 1705 may execute a sequence of machine-readable instructions, which may be embodied in a program or software. The instructions may be stored in a memory location, such as the memory 1710. The instructions may be directed to the CPU 1705, which may subsequently program or otherwise configure the CPU 1705 to implement methods of the present disclosure. Examples of operations performed by the CPU 1705 may include fetch, decode, execute, and/or writeback.

The CPU 1705 may be part of a circuit, such as an integrated circuit. One or more other components of the system 1701 may be included in the circuit. In some cases, the circuit is an application specific integrated circuit (ASIC).

The storage unit 1715 may store files, such as drivers, libraries and/or saved programs. The storage unit 1715 may store user data, e.g., user preferences and/or user programs. The computer system 1701 in some cases may include one or more additional data storage units that are external to the computer system 1701, such as located on a remote server that is in communication with the computer system 1701 through an intranet or the Internet.

The computer system 1701 may communicate with one or more remote computer systems through the network 1730. For instance, the computer system 1701 may communicate with a remote computer system of a user (e.g., a fluorescence imaging instrument, a microscope, a fluorescence spectrometer). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 1701 via the network 1730.

Methods as described herein may be implemented by way of machine (e.g., computer processor) executable code stored on an electronic storage location of the computer system 1701, such as, for example, on the memory 1710 or electronic storage unit 1715. The machine executable or machine-readable code may be provided in the form of software. During use, the code may be executed by the processor 1705. In some cases, the code may be retrieved from the storage unit 1715 and stored on the memory 1710 for ready access by the processor 1705. In some situations, the electronic storage unit 1715 may be precluded, and/or machine-executable instructions are stored on memory 1710.

The code may be pre-compiled and/or configured for use with a machine having a processer adapted to execute the code or may be compiled during runtime. The code may be supplied in a programming language that may be selected to enable the code to execute in a pre-compiled or as-compiled fashion.

Aspects of the systems and methods provided herein, such as the computer system 1701, may be embodied in programming Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and/or electromagnetic waves, such as used across physical interfaces between local devices, through wired and/or optical landline networks and/or over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and/or fiber optics, including the wires that comprise a bus within a computer system. Carrier-wave transmission media may take the form of electric or electromagnetic signals, or acoustic or light waves such as those generated during radio frequency (RF) and/or infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1701 may include or be in communication with an electronic display 1735 that comprises a user interface (UI) 1740 for providing, for example, orders and/or options for controlling the parameters (e.g., time duration, intensity of energy source, type of energy source) of photobleaching or photocleaving, Examples of UI's include, without limitation, a graphical user interface (GUI) and/or web-based user interface.

Methods and systems of the present disclosure may be implemented by way of one or more algorithms. An algorithm may be implemented by way of software upon execution by the central processing unit 1705. The algorithm may, for example, implement parts of methods described herein.

### EXAMPLES

### Example 1: Assay for Counting of Monomers in Individual Oligomeric Complex

Sandwich ELISA assay is performed for counting of monomers in each individual oligomeric complex (FIGS. 1A and 1B). A biological fluid (e.g., CSF, blood, saliva) comprising alpha-synuclein oligomers 103 is introduced on to a glass slide 101 functionalized with a common alpha-synuclein specific antibody 102. The detecting secondary antibody 104, labeled with a single fluorophore 105 is then flowed through resulting in a single molecule sandwich ELISA assay. Oligomeric complex 106 will bind to multiple secondary antibodies. Eliminating individual fluorophores such as by photobleaching individual fluorescent spots 110 may result in individual intensity trace and/or step drops in fluorescent intensity 120. Each step drops in fluorescent intensity 121 correlates to a photo destruction of a fluorophore and the number of steps corresponds to the total number of fluorophores or the secondary antibody. By collating the number of step-drops per individual oligomeric complex, a statistical change in monomer distributions of the oligomeric complex at single molecule sensitivity may be observed 130. For example, this assay may be used to provide a frequency distribution of the monomer counts across millions of captured alpha-synuclein species. A significant shift in this distribution may indicate an abnormality. The significant shift in this distribution may provide a quantitative measurement for detecting a disease or disorder.

### Example 2: Assay for Selecting a Secondary Antibody

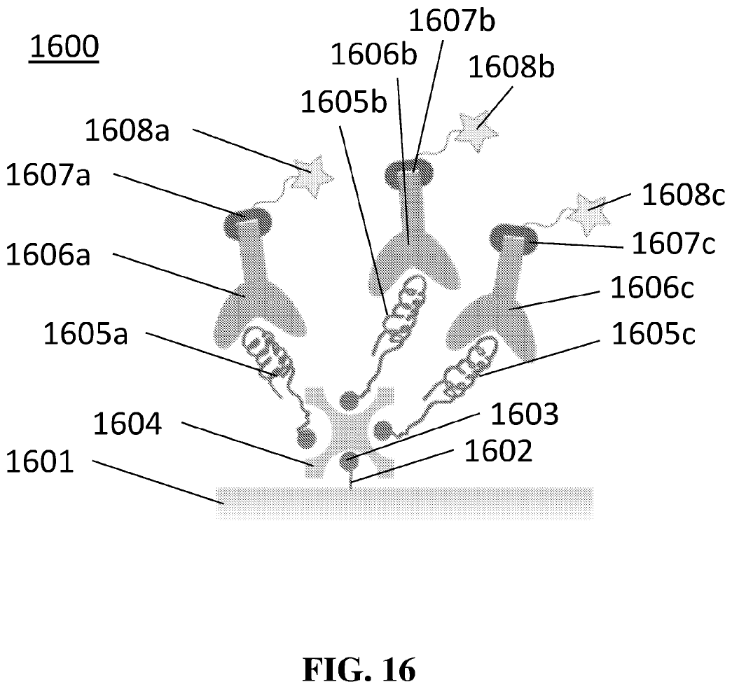

In order to count multiple monomers in the complex (e.g., in alpha-synuclein), each monomer may be bound by a single secondary antibody. As such, a set of parameters in the secondary antibody can be selected for comprising (a) steric hindrance due to size and/or shape of secondary antibody affecting access and/or binding to closely packed monomers (e.g., epitopes), (b) effect on the binding affinity of the secondary antibody due to conjugation of a fluorophore and/or (c) non-specific hydrophobic interactions between fluorophores. FIG. 16 shows an assay where recombinant alpha-synuclein is trimerized with streptavidin and immobilized on a functionalized surface. The assay may be used to select fluorescently labeled antibody (e.g., via Protein G label) for specific and strong binding. A tetramerized streptavidin 1604 and biotin conjugated alpha-synuclein 1605a-c is used to select antibodies 1606a-c coupled with fluorescent labels 1608 a-c via Protein G label 1607a-c that show specific and strong binding. The assay 1600 may be configured to select against crowding effects of fluorescently labeled secondary antibody. The assay 1600 may be used to image and measure the fluorescent drop counts after incubating it with labeled secondary antibody and thus rapidly test different secondary antibodies and the washing and imaging conditions to recapitulate the binding of the secondary antibodies.

N-terminal labeling of protein G and binding with secondary antibodies: A water soluble and positively charged fluorophore such as Atto647N or rhodamine may be conjugated to pyridine carboxaldehyde (PCA) functional group via a long PEG(10) linker to produce the PCA-fluorophore reagent. This PCA reagent may be conjugated to protein G and purified. This singly labeled protein G reagent 1607 may then be used to label all secondary antibodies 1606 via binding to the Fc segment as illustrated in FIG. 16. The assay 1600 may be used to select antibodies for effective labeling of the oligomers (e.g., one single antibody per monomer) from a mixture of various antibodies.

Streptavidin mediated surface multimerization of alpha-synuclein: Streptavidin may be mixed with 4 equivalents of biotinylated alpha-synuclein and incubated on the biotin-functionalized PEG passivated surface to produce a streptavidin cluster that may contain three alpha-synuclein proteins. The complexity and the multimerization of the streptavidin complex may be further increased two-fold by mixing a sub-stoichiometric equivalent (e.g., 0.25-0.5 equivalents) of a bifunctional biotin PEG linker with the biotinylated alpha-synuclein.

Rapid screening of fluorescently bound secondary antibody to recapitulate multimeric state of the surface bound alpha-synuclein: After incubating the fluorescently bound secondary antibody with the multimerized alpha-synuclein on the surface, images may be obtained using an imaging system (e.g., confocal fluorescent microscope). The mixture (e.g., sample) may be subjected to imaging conditions comprising a buffer, an energy source (e.g., laser), and a camera to photo bleach the sample. The intensity trace associated with each fluorescent spot may then be measured. Using the diffraction-limited nature of the microscope, at sufficient dilution, each fluorescent spot may be the fluorescence signal from a single streptavidin/alpha-synuclein/secondary antibody complex. The step drop counts for every individual spot may be correlated with the expected distribution (median counts) of alpha-synuclein on every streptavidin protein. This assay for screening setup may be streamlined and along with the single correlation score as the comparison metric, the assay may be used to substantially rapidly select a secondary antibody. The secondary antibody identified by the assay 1600 may be selective for the target protein (e.g., alpha-synuclein), may not sterically hinder other binding events to the same complex, and may demonstrate high affinity for binding in the presence of fluorescently tagged protein G. A polypeptide that may be frequently present in a sample may be used as a negative control in assay 1600 to ensure that the identified secondary antibody has high selectivity for the target protein. A biotinylated albumin protein, one of the most abundant proteins in the CSF, may be used as a negative control to ensure the selected antibody has high selectivity for alpha-synuclein.

### Example 3: Slide Passivation

The effect of slide passivation for single molecule imaging studies is critical to prevent non-specific binding of fluorescently labeled biomolecules. An experiment to improve glass slide passivation was performed under the hypothesis that the optimal ratio of inert silane (methyltrimethoxysilane) with biotinylated silane (silane PEG Biotin) provides for adequate functional sites in a background of inert regions. The interaction between streptavidin-biotin conjugated to the surface with the fluorescent Atto647N-biotin was studied and contrasted with Atto647N-biotin interacting non-specifically with the surface.

Experimental method: (a) Slide preparation: A glass slide (45 mm) from Bioptechs Inc optimal slide was prepared by mixing biotinylated silane and the inert silane at different molar ratio (1:1 to 1:50) in a solution of methanol/2% acetic acid. The slide was then rinsed with nanopure water and cured at 110° C. in a vacuum oven. A negative control (without the biotinylated silane) was also prepared. (b) Fluorescent biomolecule immobilization: The slide was immersed in Streptavidin (2 nM) in 0.1 M phosphate buffer (pH 7.5) for 10 minutes to complete the reaction of biotin with streptavidin. The slide was washed with water, followed up with 2 pM of Atto647N-biotin in phosphate buffer (pH 7.5) and incubated for 30 mins. The slide was washed and imaged. (c) Total Internal Fluorescent microscopy imaging: The glass slide was assembled into an FCS2 Bioptechs chamber and imaged with a Nikon Ti-E inverted TIRF microscope, equipped with 405, 488, 561 and 647 nm laser, 60×1.49 NA oil objective and iXon EMCCD camera. The Atto647N coupled slide was imaged, and single fluorescent molecules were counted on the experimental slide and inert slide using custom image processing scripts.

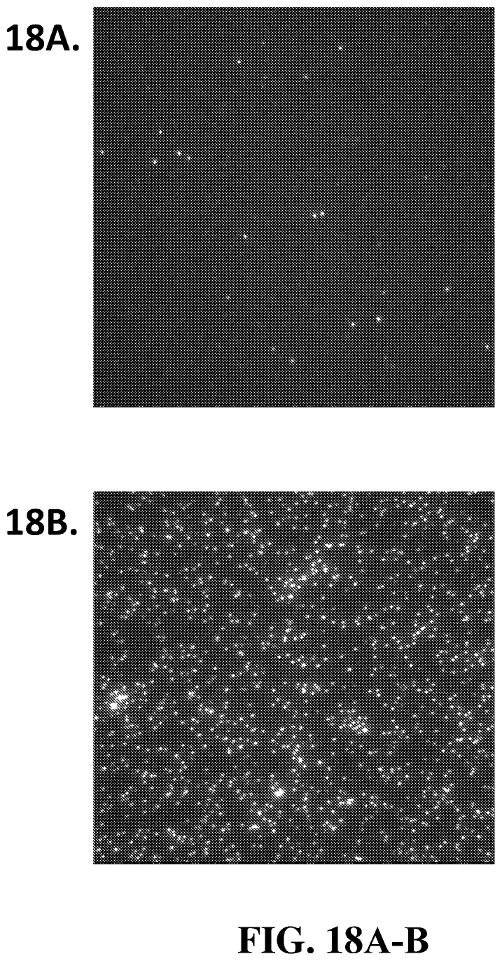

The optimal molar ratio of biotinylated/inert silane was found to be 1:20. As seen in FIG. 18A-B (150 μm×150 μm imaging micrograph), the counts of fluorescent biomolecule can be contrasted between the two slides (inert, left panel) and biotinylated surface (right panel). FIG. 18A-18B show the effect of slide passivation indicating the low non-specific level of multimerized streptavidin/Atto647N-biotin complex. The counts of 5:1 of Atto647N-biotin:Streptavidin ratio (FIG. 18B) contrasting to the low counts of Atto647N-biotin (FIG. 18A) immobilized on the surface through interactions of biotin with streptavidin complex on surface can be clearly seen.

### Example 4: Analyzing Trimerized Streptavidin/Alpha-Synuclein

Streptavidin molecules have four sites for biotin binding. With one site occupied through binding of surface biotin, there are three potential vacant sites available for Alpha-synuclein-biotin binding. The goal of the experiment was to form a trimerized alpha-synuclein by optimizing incubation times and concentrations of alpha-synuclein, a secondary labeled antibody.

Experimental methods: (a) Streptavidin-alpha-synuclein coupling on slide: The passivated biotinylated slide (as described in Example 3) was used to couple streptavidin on the surface. Biotinylated alpha-synuclein was incubated at varying concentrations from 1:1 to 1:100 streptavidin to biotinylated alpha-synuclein. The slides were washed with 1% Tween in phosphate buffer to remove excess unreacted alpha-synuclein. (b) Detector antibody binding: The detector antibody labeled with Alexa-488 was incubated on the slide for 30 minutes. Multiple washes with phosphate buffer (0.1% Tween, 50 mM NaCl) were performed to remove excess antibody. (c) Imaging Using the TIRF microscope setup as described in Example 3, the image files were photobleached by irradiating the slides with a 488 laser for 2 minutes with 1 sec acquisition. (d) Image processing: Each individual spot was analyzed using custom image analyzing scripts and the fluorescent intensity measured through time. Step drops in intensity corresponded to a photobleaching event, which in turn correlated to the presence of a biomolecule. A three step drop in intensity indicated the presence of three fluorophores, e.g., 3 alpha-synuclein binding. A histogram of counts of molecules with different number of step-drops was plotted.

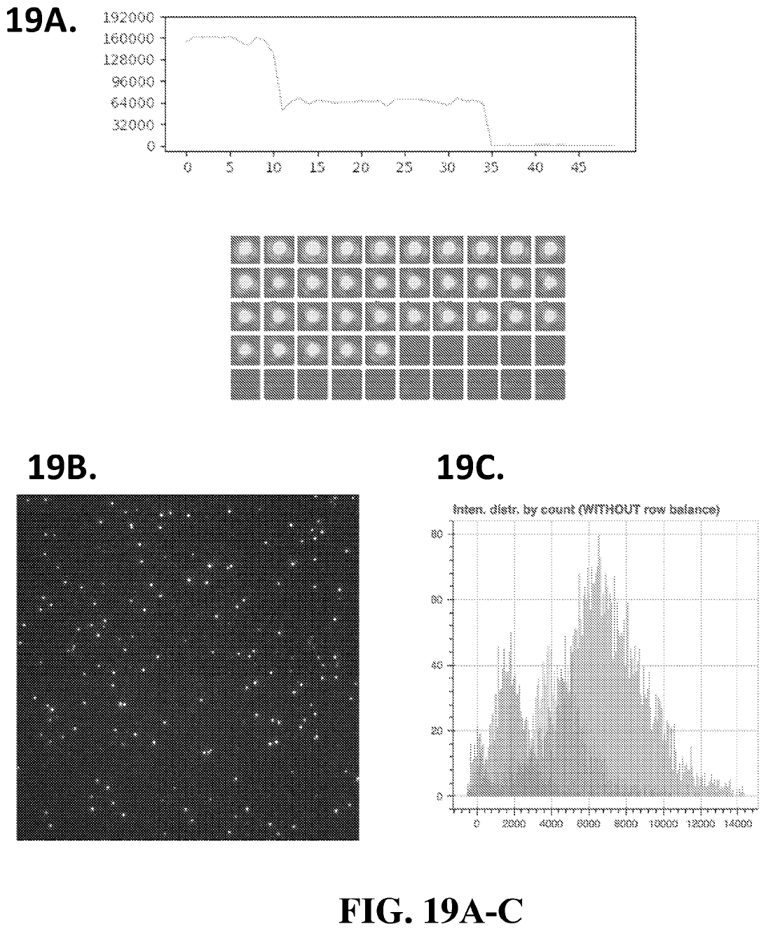

The data showed that a molar ratio of 1:50 streptavidin/alpha-synuclein biotin was optimal in producing maximal three step drop molecules. FIG. 19A-19C show photobleaching and image processing algorithms performed on trimerized streptavidin/alpha-synuclein biotin with detection antibody indicated a three-count data. FIG. 19A shows the photobleaching trace of a single peptide molecule. FIG. 19B shows a representative field of single molecules of Streptavidin/Alpha-synuclein-biotin coupled to a second fluorescently labeled detector antibody (labeled with Alexa647) to form a complex. FIG. 19C shows the distribution of the counts/intensity towards a three-count data, as indicated by the histogram on the right. The data show that the maximal count of 3 or the trimerized alpha-synuclein with streptavidin was observed.

While preferred embodiments have been shown and described herein, it will be obvious to those skilled in the art that such embodiments are provided by way of example only. It is not intended that the reagents, compositions, systems, and methods of the present disclosure be limited by the specific examples provided within the specification. While a variety of embodiments have been described with reference to the aforementioned specification, the descriptions and illustrations of the embodiments herein are not meant to be construed in a limiting sense. Numerous variations, changes, and substitutions will now occur to those skilled in the art without departing from the enabling scope of the present disclosure. Furthermore, it shall be understood that all aspects of the present disclosure are not limited to the specific depictions, configurations or relative proportions set forth herein which depend upon a variety of conditions and variables. It may be understood that various alternatives to the embodiments of the present disclosure may be employed in practicing the disclosed concepts. It is therefore contemplated that the scope of the enabling disclosure shall also cover any such alternatives, modifications, variations or equivalents. It is intended that the following claims define the scope of the disclosure and that methods and structures within the scope of these claims and their equivalents be covered thereby.

### EMBODIMENTS

The following non-limiting embodiments provide illustrative examples of the invention, but do not limit the scope of the invention.

Embodiment 1. A method for analyzing a polypeptide complex from a subject, comprising: (a) providing the polypeptide complex coupled to a capture unit immobilized to a support, wherein the polypeptide complex comprises a plurality of polypeptide molecules; (b) coupling one or more reporter moieties to the polypeptide complex, wherein the one or more reporter moieties comprises a plurality of detectable labels; (c) detecting one or more signals from the plurality of detectable labels; and (d) subjecting the plurality of detectable labels to conditions sufficient to render at most a subset of the one or more detectable labels undetectable.

Embodiment 2. The method of embodiment 1, further comprising (e) detecting a disease or disorder in the subject based at least in part on the one or more signals detected in (c).

Embodiment 3. The method of embodiment 1 or 2, further comprising repeating (c) and (d) at least once until no signal is detected from the polypeptide complex.

Embodiment 4. The method of any one of embodiments 1-3, wherein at least a subset of the plurality of polypeptide molecules in the polypeptide complex is quantified.

Embodiment 5. The method of any one of embodiments 1-4, wherein a reporter moiety of the one or more reporter moieties is coupled to a polypeptide molecule of the plurality of polypeptide molecules.

Embodiment 6. The method of any one of embodiments 1-5, wherein a polypeptide molecule of the plurality of polypeptide molecules comprises one or more binding units, wherein at least one binding unit of the one or more binding units is coupled to a reporter moiety of the one or more reporter moieties.

Embodiment 7. The method of any one of embodiments 1-6, wherein a reporter moiety of the one or more reporter moieties comprises one or more recognition units coupled to at least a subset of the plurality of polypeptide molecules.

Embodiment 8. The method of embodiment 7, wherein the reporter moiety comprises a spacer coupled to a detectable label of the one or more detectable labels.

Embodiment 9. The method of any one of embodiments 1-8, wherein the one or more signals correspond to the plurality of detectable labels.

Embodiment 10. The method of embodiment 8, wherein the spacer adjoins the detectable label and the recognition unit.

Embodiment 11. The method of any one of embodiments 1-10, wherein (d) comprises photobleaching a detectable label of the one or more detectable labels.

Embodiment 12. The method of any one of embodiments 1-10, wherein (d) comprises removing a detectable label of the one or more detectable labels from the polypeptide complex.

Embodiment 13. The method of any one of embodiments 1-12, wherein the polypeptide complex comprises at least 2 polypeptide molecules.

Embodiment 14. The method of any one of embodiments 1-12, wherein the polypeptide complex comprises at least 5 polypeptide molecules.

Embodiment 15. The method of any one of embodiments 1, wherein the polypeptide complex comprises at least 10 polypeptide molecules.

Any one of embodiments 16. The method of any one of embodiments 1, wherein the polypeptide complex comprises at least 20 polypeptide molecules.

Embodiment 17. The method of any one of embodiments 1-16, wherein the capture unit comprises no more than one antibody.

Embodiment 18. The method of any one of embodiments 1-17, wherein the polypeptide complex is a biomarker.

Embodiment 19. The method of embodiment 18, wherein an expression level of the biomarker is indicative of a disease or disorder.

Embodiment 20. The method of embodiment 19, wherein the disease or disorder is Parkinson's disease (PD), Parkinson's disease with dementia (PDD), dementia with Lewy bodies (DLB), multiple system atrophy (MSA), Alzheimer's disease (AD), Pick's disease, frontotemporal dementia (FTD), traumatic brain injury, chronic traumatic encephalopathy (CTE), Huntington's disease, fragile X syndrome, amyotrophic lateral sclerosis (ALS), cryoglobulinemia, amyloidosis, prion disease, transmissible spongiform encephalopathy, or Creutzfeldt-Jakob Disease.

Embodiment 21. The method of embodiment 18, wherein the biomarker is an amyloid protein, an amyloid fibril, an amyloid beta, an amyloid precursor protein, a tau protein, a microtubule-associated protein tau, an alpha synuclein, an immunoglobulin, an islet amyloid polypeptide, a huntingtin protein, a FMRP, a polyglutamine repeat protein, a dipeptide repeat protein, a TDP-43, matrin-3, or a prion.

Embodiment 22. The method of any one of embodiments 18-21, wherein the biomarker corresponds to a neurodegenerative disease or disorder.

Embodiment 23. The method of any one of embodiments 18-21, wherein the expression level of the biomarker is quantified and correlated to a health assessment.

Embodiment 24. The method of any one of embodiments 1-23, wherein (a) comprises providing the polypeptide complex from a sample from the subject.

Embodiment 25. The method of embodiment 24, wherein the sample comprises cerebrospinal fluid, brain homogenate, tissue homogenate, tissue extract, cell extract, cell homogenate, cell lysate, whole blood, plasma, serum, bodily waste or excretion, or any combination thereof.

Embodiment 26. The method of any one of embodiments 1-25, wherein the subject's health is assessed based on the detection of the one or more signals detected in (c).

Embodiment 27. The method of any one of embodiments 1-26, wherein the support is a bead, a polymer matrix, or an array.

Embodiment 28. The method of embodiment 27, wherein the array is a microscopic slide.

Embodiment 29. The method of any one of embodiments 1-28, wherein the capture unit is immobilized directly to the support.

Embodiment 30. The method of any one of embodiments 1-29, wherein (c) or (d) further comprises providing an energy source.

Embodiment 31. The method of any one of embodiments 1-30, wherein (c) comprises providing a first energy source sufficient to render the one or more detectable labels optically detectable.

Embodiment 32. The method of embodiment 31, wherein the one or more detectable labels emit an optical signal.

Embodiment 33. The method of embodiment 32, wherein the optical signal is a fluorescent signal.

Embodiment 34. The method of embodiment 31, wherein the first energy source is a light or a laser.

Embodiment 35. The method of any one of embodiments 1-34, wherein (d) comprises providing a second energy source sufficient to render the at most a subset of the one or more detectable labels undetectable.

Embodiment 36. The method of embodiment 35, wherein the second energy source is a light or a laser.

Embodiment 37. The method of any one of any one of embodiments 31-36, wherein the first energy source and the second energy source are the same energy source.

Embodiment 38. The method of any one of embodiments 1-37, wherein the plurality of polypeptide molecules is homogenous.

Embodiment 39. The method of any one of embodiments 1-37, wherein the plurality of polypeptide molecules is heterogeneous.

Embodiment 40. The method of any one of embodiments 1-39, wherein the capture unit is coupled to either the polypeptide complex or an individual polypeptide molecule of the polypeptide complex.

Embodiment 41. The method of any one of embodiments 1-40, wherein the polypeptide complex is coupled to the capture unit via a cross-linker.

Embodiment 42. The method of embodiment 41, wherein the cross-linker is an amine specific cross-linker.

Embodiment 43. The method of embodiment 41, wherein the cross-linker is a PEG linker.

Embodiment 44. The method of embodiment 43, wherein the PEG linker is a 1-10 kDa PEG linker.

Embodiment 45. The method of embodiment 43, wherein the PEG linker is a bifunctional biotin PEG linker.

Embodiment 46. The method of any one of embodiments 1-45, wherein the method further comprises determining a frequency of polypeptide molecule counts based at least in part on the one or more signals detected in (c).

Embodiment 47. The method of embodiment 46, wherein the method further comprises detecting the disease or disorder in the subject based at least in part on a shift in a distribution of the frequency of polypeptide molecule counts.

Embodiment 48. The method of any one of claim 1-10, 12-30 or 38-48, wherein the conditions sufficient to render at most a subset of the one or more detectable labels undetectable comprises dye quenching.

Embodiment 49. The method of any one of embodiments 1-10, 12-30 or 38-48, wherein the conditions sufficient to render at most a subset of the one or more detectable labels undetectable comprises enzymatic cleavage of the one or more detectable labels.

Embodiment 50. A method for analyzing a polypeptide complex from a subject, comprising: (a) providing the polypeptide complex and one or more reporter moieties coupled thereto, wherein the one or more reporter moieties comprises a plurality of detectable labels, wherein the polypeptide complex comprises a plurality of polypeptide molecules; (b) detecting one or more signals from the plurality of detectable labels; and (c) subjecting the one or more detectable labels to conditions sufficient to render at most a subset of the one or more detectable labels undetectable.

Embodiment 51. The method of embodiment 50, further comprising (d) using at least the one or more signals to quantify an amount of the plurality of polypeptide molecules in the polypeptide complex.

Embodiment 52. The method of embodiment 51, further comprising repeating (b) and (c) at least once until no signal is detected from the polypeptide complex.

Embodiment 53. The method of any one of embodiments 50-52, wherein at least a subset of the plurality of polypeptide molecules in the polypeptide complex is quantified.

Embodiment 54. The method of any one of embodiments 50-53, wherein a reporter moiety of the one or more reporter moieties is coupled to a polypeptide molecule of the plurality of polypeptide molecules.

Embodiment 55. The method of any one of embodiments 50-54, wherein a polypeptide molecule of the plurality of polypeptide molecules comprises one or more binding units, wherein at least one binding unit of the one or more binding units is coupled to a reporter moiety of the one or more reporter moieties.

Embodiment 56. The method of any one of embodiments 50-55, wherein a reporter moiety of the one or more reporter moieties comprises one or more recognition units coupled to at least a subset of the plurality of polypeptide molecules.

Embodiment 57. The method of embodiment 56, wherein the reporter moiety comprises a spacer coupled to a detectable label of the one or more detectable labels.

Embodiment 58. The method of any one of embodiments 50-57, wherein the one or more signals correspond to the plurality of detectable labels.

Embodiment 59. The method of embodiment 57, wherein the spacer adjoins the detectable label and the recognition unit.

Embodiment 60. The method of any one of embodiments 50-59, wherein (c) comprises photobleaching a detectable label of the one or more detectable labels.

Embodiment 61. The method of any one of embodiments 50-59, wherein (c) comprises removing a detectable label of the one or more detectable labels from the polypeptide complex.

Embodiment 62. The method of any one of embodiments 50-61, wherein the polypeptide complex comprises at least 2 polypeptide molecules.

Embodiment 63. The method of any one of embodiments 50-61, wherein the polypeptide complex comprises at least 5 polypeptide molecules.

Embodiment 64. The method of any one of embodiments 50-61, wherein the polypeptide complex comprises at least 10 polypeptide molecules.

Embodiment 65. The method of any one of embodiments 50-61, wherein the polypeptide complex comprises at least 20 polypeptide molecules.

Embodiment 66. The method of any one of embodiments 50-65, wherein the capture unit comprises no more than one antibody.

Embodiment 67. The method of any one of embodiments 51-66, further comprising (e) detecting a disease or disorder in the subject based at least in part on the one or more signals detected in (c).

Embodiment 68. The method of any one of embodiments 50-67, wherein the polypeptide complex is a biomarker.

Embodiment 69. The method of embodiment 68, wherein an expression level of the biomarker is indicative of a disease or disorder.

Embodiment 70. The method of embodiment 69, wherein the disease or disorder is Parkinson's disease (PD), Parkinson's disease with dementia (PDD), dementia with Lewy bodies (DLB), multiple system atrophy (MSA), Alzheimer's disease (AD), Pick's disease, frontotemporal dementia (FTD), traumatic brain injury, chronic traumatic encephalopathy (CTE), Huntington's disease, fragile X syndrome, amyotrophic lateral sclerosis (ALS), cryoglobulinemia, amyloidosis, prion disease, transmissible spongiform encephalopathy, or Creutzfeldt-Jakob Disease.

Embodiment 71. The method of embodiment 68 or 69, wherein the biomarker is an amyloid protein, an amyloid fibril, an amyloid beta, an amyloid precursor protein, a tau protein, a microtubule-associated protein tau, an alpha synuclein, an immunoglobulin, an islet amyloid polypeptide, a huntingtin protein, a FMRP, a polyglutamine repeat protein, a dipeptide repeat protein, a TDP-43, matrin-3, or a prion.

Embodiment 72. The method of embodiment 68 or 69, wherein the biomarker corresponds to a neurodegenerative disease or disorder.

Embodiment 73. The method of embodiment 68 or 69, wherein the expression level of the biomarker is quantified and correlated to a health assessment.

Embodiment 74. The method of any one of embodiments 50-73, wherein (a) comprises providing the polypeptide complex from a sample from a subject.

Embodiment 75. The method embodiment 74, wherein the sample comprises cerebrospinal fluid, brain homogenate, tissue homogenate, tissue extract, cell extract, cell homogenate, cell lysate, whole blood, plasma, serum, bodily waste or excretion, or any combination thereof.

Embodiment 76. The method of any one of embodiments 50-74, wherein the subject's health is assessed based on the detection of the one or more signals detected in (b).

Embodiment 77. The method of any one of embodiments 50-76, wherein the polypeptide complex is coupled to a capture unit immobilized to a support.

Embodiment 78. The method of any one of embodiments 50-77, wherein the support is a bead, a polymer matrix, or an array.

Embodiment 79. The method of embodiment 78, wherein the array is a microscopic slide.

Embodiment 80. The method of embodiment 77, wherein the capture unit is immobilized directly to the support.

Embodiment 81. The method of any one of embodiments 50-80, wherein (b) and (c) further comprises providing an energy source.

Embodiment 82. The method of any one of embodiments 50-81, wherein (b) comprises providing a first energy source sufficient to render the one or more detectable labels optically detectable.

Embodiment 83. The method of embodiment 82, wherein the one or more detectable labels emit an optical signal.

Embodiment 84. The method of embodiment 83, wherein the optical signal is a fluorescent signal.

Embodiment 85. The method of embodiment 82, wherein the first energy source is a light or a laser.

Embodiment 86. The method of any one of embodiments 50-81, wherein (c) comprises providing a second energy source sufficient to render the at most a subset of the one or more detectable labels undetectable.

Embodiment 87. The method of embodiment 86, wherein the second energy source is a light or a laser.

Embodiment 88. The method of any one of any one of embodiments 82-87, wherein the first energy source and the second energy source are the same energy source.

Embodiment 89. The method of any one of embodiments 50-88, wherein the plurality of polypeptide molecules is homogenous.

Embodiment 90. The method of any one of embodiments 50-88, wherein the plurality of polypeptide molecules is heterogeneous.

Embodiment 91. The method of any one of embodiments 50-90, wherein the capture unit is coupled to either the polypeptide complex or an individual polypeptide molecule of the polypeptide complex.

Embodiment 92. The method of any one of embodiments 50-91, wherein the polypeptide complex is coupled to the capture unit via a cross-linker.

Embodiment 93. The method of embodiment 92, wherein the cross-linker is an amine specific cross-linker.

Embodiment 94. The method of embodiment 92, wherein the cross-linker is a PEG linker.

Embodiment 95. The method of embodiment 94, wherein the PEG linker is a 1-10 kDa PEG linker.

Embodiment 96. The method of embodiment 94, wherein the PEG linker is a bifunctional biotin PEG linker.

Embodiment 97. The method of any one of embodiments 50-96, wherein the method further comprises determining a frequency of polypeptide molecule counts based at least in part on the one or more signals detected in (b).

Embodiment 98. The method of any one of embodiments 50-97, wherein the method further comprises detecting a disease or disorder in the subject based at least in part on a shift in a distribution of the frequency of polypeptide molecule counts.

Embodiment 99. The method of any one of embodiments 50-59, 61-80, and 89-98, wherein the conditions sufficient to render at most a subset of the one or more detectable labels undetectable comprises dye quenching.

Embodiment 100. The method of any one of embodiments 50-59, 61-80, and 89-98, wherein the conditions sufficient to render at most a subset of the one or more detectable labels undetectable comprises enzymatic cleavage of the one or more detectable labels.

Embodiment 101. A method for analyzing a polypeptide complex comprising a plurality of polypeptides of a subject at a single molecule level, comprising detecting an individual polypeptide of the plurality of polypeptides at a sensitivity of at least 60%.

Embodiment 102. A method for analyzing a polypeptide complex comprising a plurality of polypeptides of a subject at a single molecule level, comprising detecting an individual polypeptide of the plurality of polypeptides at a sensitivity of at least 60%.

What is claimed is:

1. A method for analyzing a polypeptide complex from a subject, comprising:
(A) providing said polypeptide complex coupled to a capture unit immobilized to a support, wherein said polypeptide complex comprises a plurality of polypeptide molecules;
(B) coupling one or more reporter moieties to said polypeptide complex, wherein said one or more reporter moieties comprises a plurality of detectable labels;
(C) detecting one or more signals from said plurality of detectable labels; and
(D) subjecting said plurality of detectable labels to conditions sufficient to render at most a subset of said one or more detectable labels undetectable.

2. The method of claim 1, further comprising (E) detecting a disease or disorder in said subject based at least in part on said one or more signals detected in (C).

3. The method of claim 1, further comprising repeating (C) and (D) at least once until no signal is detected from said polypeptide complex.

4. The method of claim 3, wherein at least a subset of said plurality of polypeptide molecules in said polypeptide complex is quantified.

5. The method of claim 1, wherein a reporter moiety of said one or more reporter moieties is coupled to a polypeptide molecule of said plurality of polypeptide molecules.

6. The method of claim 1, wherein a polypeptide molecule of said plurality of polypeptide molecules comprises one or more binding units, wherein at least one binding unit of said one or more binding units is coupled to a reporter moiety of said one or more reporter moieties.

7. The method of claim 1, wherein a reporter moiety of said one or more reporter moieties comprises one or more recognition units coupled to at least a subset of said plurality of polypeptide molecules.

8. The method of claim 7, wherein said reporter moiety comprises a spacer coupled to a detectable label of said one or more detectable labels.

9. The method of claim 1, wherein said one or more signals correspond to said plurality of detectable labels.

10. The method of claim 8, wherein said spacer adjoins said detectable label and said recognition unit.

11. The method of claim 1, wherein (D) comprises photobleaching a detectable label of said one or more detectable labels.

12. The method of claim 1, wherein (D) comprises removing a detectable label of said one or more detectable labels from said polypeptide complex.

13. The method of claim 1, wherein said polypeptide complex comprises at least 2 polypeptide molecules.

14. The method of claim 1, wherein said polypeptide complex comprises at least 5 polypeptide molecules.

15. The method of claim 1, wherein said polypeptide complex comprises at least 10 polypeptide molecules.

16. The method of claim 1, wherein said polypeptide complex comprises at least 20 polypeptide molecules.

17. The method of claim 1, wherein said capture unit comprises no more than one antibody.

18. The method of claim 1, wherein said polypeptide complex is a biomarker.

19. The method of claim 18, wherein an expression level of said biomarker is indicative of a disease or disorder.

20. The method of claim 19, wherein said disease or disorder is Parkinson's disease (PD), Parkinson's disease with dementia (PDD), dementia with Lewy bodies (DLB), multiple system atrophy (MSA), Alzheimer's disease (AD), Pick's disease, frontotemporal dementia (FTD), traumatic brain injury, chronic traumatic encephalopathy (CTE), Huntington's disease, fragile X syndrome, amyotrophic lateral sclerosis (ALS), cryoglobulinemia, amyloidosis, prion disease, transmissible spongiform encephalopathy, or Creutzfeldt-Jakob Disease.

21. The method of claim 18, wherein said biomarker is an amyloid protein, an amyloid fibril, an amyloid beta, an amyloid precursor protein, a tau protein, a microtubule-associated protein tau, an alpha synuclein, an immunoglobulin, an islet amyloid polypeptide, a huntingtin protein, a FMRP, a polyglutamine repeat protein, a dipeptide repeat protein, a TDP-43, matrin-3, or a prion.

22. The method of claim 18, wherein said biomarker corresponds to a neurodegenerative disease or disorder.

23. The method of claim 18, wherein said expression level of said biomarker is quantified and correlated to a health assessment.

24. The method of claim 1, wherein (a) comprises providing said polypeptide complex from a sample from said subject.

25. The method of claim 24, wherein said sample comprises cerebrospinal fluid, brain homogenate, tissue homogenate, tissue extract, cell extract, cell homogenate, cell lysate, whole blood, plasma, serum, bodily waste or excretion, or any combination thereof.

26. The method of claim 24, wherein said subject's health is assessed based on said detection of said one or more signals detected in (C).

27. The method of claim 1, wherein said support is a bead, a polymer matrix, or an array.

28. The method of claim 27, wherein said array is a microscopic slide.

29. The method of claim 1, wherein said capture unit is immobilized directly to said support.

30. The method of claim 1, wherein (C) or (D) further comprises providing an energy source.

31. The method of claim 30, wherein (C) comprises providing a first energy source sufficient to render said one or more detectable labels optically detectable.

32. The method of claim 31, wherein said one or more detectable labels emit an optical signal.

33. The method of claim 32, wherein said optical signal is a fluorescent signal.

34. The method of claim 31, wherein said first energy source is a light or a laser.

35. The method of claim 30, wherein (D) comprises providing a second energy source sufficient to render said at most a subset of said one or more detectable labels undetectable.

36. The method of claim 35, wherein said second energy source is a light or a laser.

37. The method of any one of claims 31-36, wherein said first energy source and said second energy source are the same energy source.

38. The method of claim 1, wherein said plurality of polypeptide molecules is homogenous.

39. The method of claim 1, wherein said plurality of polypeptide molecules is heterogeneous.

40. The method of claim 1, wherein said capture unit is coupled to either said polypeptide complex or an individual polypeptide molecule of said polypeptide complex.

41. The method of claim 1, wherein said polypeptide complex is coupled to said capture unit via a cross-linker.

42. The method of claim 41, wherein said cross-linker is an amine specific cross-linker.

43. The method of claim 41, wherein said cross-linker is a PEG linker.

44. The method of claim 43, wherein said PEG linker is a 1-10 kDa PEG linker.

45. The method of claim 43, wherein said PEG linker is a bifunctional biotin PEG linker.

46. The method of claim 1, wherein said method further comprises determining a frequency of polypeptide molecule counts based at least in part on said one or more signals detected in (C).

47. The method of claim 46, wherein said method further comprises detecting said disease or disorder in said subject based at least in part on a shift in a distribution of said frequency of polypeptide molecule counts.

48. The method of claim 1, wherein the conditions sufficient to render at most a subset of said one or more detectable labels undetectable comprises dye quenching.

49. The method of claim 1, wherein the conditions sufficient to render at most a subset of said one or more detectable labels undetectable comprises enzymatic cleavage of said one or more detectable labels.

50. A method for analyzing a polypeptide complex from a subject, comprising:
(A) providing said polypeptide complex and one or more reporter moieties coupled thereto, wherein the one or more reporter moieties comprises a plurality of detectable labels, wherein the polypeptide complex comprises a plurality of polypeptide molecules;
(B) detecting one or more signals from said plurality of detectable labels; and
(C) subjecting said one or more detectable labels to conditions sufficient to render at most a subset of said one or more detectable labels undetectable.

51. The method of claim 50, further comprising (D) using at least said one or more signals to quantify an amount of said plurality of polypeptide molecules in said polypeptide complex.

52. The method of claim 51, further comprising repeating (B) and (C) at least once until no signal is detected from said polypeptide complex.

53. The method of claim 52, wherein at least a subset of said plurality of polypeptide molecules in said polypeptide complex is quantified.

54. The method of claim 0, wherein a reporter moiety of said one or more reporter moieties is coupled to a polypeptide molecule of said plurality of polypeptide molecules.

55. The method of claim 0, wherein a polypeptide molecule of said plurality of polypeptide molecules comprises one or more binding units, wherein at least one binding unit of said one or more binding units is coupled to a reporter moiety of said one or more reporter moieties.

56. The method of claim 0, wherein a reporter moiety of said one or more reporter moieties comprises one or more recognition units coupled to at least a subset of said plurality of polypeptide molecules.

57. The method of claim 56, wherein said reporter moiety comprises a spacer coupled to a detectable label of said one or more detectable labels.

58. The method of claim 50, wherein said one or more signals correspond to said plurality of detectable labels.

59. The method of claim 57, wherein said spacer adjoins said detectable label and said recognition unit.

60. The method of claim 0, wherein (C) comprises photobleaching a detectable label of said one or more detectable labels.

61. The method of claim 0, wherein (C) comprises removing a detectable label of said one or more detectable labels from said polypeptide complex.

62. The method of claim 0, wherein said polypeptide complex comprises at least 2 polypeptide molecules.

63. The method of claim 0, wherein said polypeptide complex comprises at least 5 polypeptide molecules.

64. The method of claim 0, wherein said polypeptide complex comprises at least 10 polypeptide molecules.

65. The method of claim 0, wherein said polypeptide complex comprises at least 20 polypeptide molecules.

66. The method of claim 0, wherein said capture unit comprises no more than one antibody.

67. The method of claim 51, further comprising (E) detecting a disease or disorder in said subject based at least in part on said one or more signals detected in (C).

68. The method of claim 0, wherein said polypeptide complex is a biomarker.

69. The method of claim 68, wherein an expression level of said biomarker is indicative of a disease or disorder.

70. The method of claim 69, wherein said disease or disorder is Parkinson's disease (PD), Parkinson's disease with dementia (PDD), dementia with Lewy bodies (DLB), multiple system atrophy (MSA), Alzheimer's disease (AD), Pick's disease, frontotemporal dementia (FTD), traumatic brain injury, chronic traumatic encephalopathy (CTE), Huntington's disease, fragile X syndrome, amyotrophic lateral sclerosis (ALS), cryoglobulinemia, amyloidosis, prion disease, transmissible spongiform encephalopathy, or Creutzfeldt-Jakob Disease.

71. The method of claim 68, wherein said biomarker is an amyloid protein, an amyloid fibril, an amyloid beta, an amyloid precursor protein, a tau protein, a microtubule-associated protein tau, an alpha synuclein, an immunoglobulin, an islet amyloid polypeptide, a huntingtin protein, a FMRP, a polyglutamine repeat protein, a dipeptide repeat protein, a TDP-43, matrin-3, or a prion.

72. The method of claim 68, wherein said biomarker corresponds to a neurodegenerative disease or disorder.

73. The method of claim 68, wherein said expression level of said biomarker is quantified and correlated to a health assessment.

74. The method of claim 50, wherein (A) comprises providing said polypeptide complex from a sample from a subject.

75. The method of claim 74, wherein said sample comprises cerebrospinal fluid, brain homogenate, tissue homogenate, tissue extract, cell extract, cell homogenate, cell lysate, whole blood, plasma, serum, bodily waste or excretion, or any combination thereof.

76. The method of claim 74, wherein said subject's health is assessed based on said detection of said one or more signals detected in (B).

77. The method of claim 50, wherein said polypeptide complex is coupled to a capture unit immobilized to a support.

78. The method of claim 50, wherein said support is a bead, a polymer matrix, or an array.

79. The method of claim 78, wherein said array is a microscopic slide.

80. The method of claim 77, wherein said capture unit is immobilized directly to said support.

81. The method of claim 50, wherein (B) and (C) further comprises providing an energy source.

82. The method of claim 81, wherein (B) comprises providing a first energy source sufficient to render said one or more detectable labels optically detectable.

83. The method of claim 82, wherein said one or more detectable labels emit an optical signal.

84. The method of claim 83, wherein said optical signal is a fluorescent signal.

85. The method of claim 82, wherein said first energy source is a light or a laser.

86. The method of claim 81, wherein (C) comprises providing a second energy source sufficient to render said at most a subset of said one or more detectable labels undetectable.

87. The method of claim 86, wherein said second energy source is a light or a laser.

88. The method of any one of claims 82-87, wherein said first energy source and said second energy source are the same energy source.

89. The method of claim 50, wherein said plurality of polypeptide molecules is homogenous.

90. The method of claim 50, wherein said plurality of polypeptide molecules is heterogeneous.

91. The method of claim 50, wherein said capture unit is coupled to either said polypeptide complex or an individual polypeptide molecule of said polypeptide complex.

92. The method of claim 50, wherein said polypeptide complex is coupled to said capture unit via a cross-linker.

93. The method of claim 92, wherein said cross-linker is an amine specific cross-linker.

94. The method of claim 92, wherein said cross-linker is a PEG linker.

95. The method of claim 94, wherein said PEG linker is a 1-10 kDa PEG linker.

96. The method of claim 94, wherein said PEG linker is a bifunctional biotin PEG linker.

97. The method of claim 50, wherein said method further comprises determining a frequency of polypeptide molecule counts based at least in part on said one or more signals detected in (B).

98. The method of claim 50, wherein said method further comprises detecting a disease or disorder in said subject based at least in part on a shift in a distribution of said frequency of polypeptide molecule counts.

99. The method of claim 50, wherein the conditions sufficient to render at most a subset of said one or more detectable labels undetectable comprises dye quenching.

100. The method of claim 50, wherein the conditions sufficient to render at most a subset of said one or more detectable labels undetectable comprises enzymatic cleavage of said one or more detectable labels.

101. A method for analyzing a polypeptide complex comprising a plurality of polypeptides of a subject at a single molecule level, comprising detecting an individual polypeptide of said plurality of polypeptides at a sensitivity of at least 60%.
